SEQUENCE LISTING (17 sequences)

SEQ ID NO: 1 - protein, 15 aa, Artificial sequence
  Synthetic
KLTWQELYQLKYKGI

SEQ ID NO: 2 - protein, 6 aa, Artificial sequence
  Synthetic
AAAVVV

SEQ ID NO: 3 - protein, 4 aa, Artificial sequence
  Synthetic
AAVV

SEQ ID NO: 4 - protein, 4 aa, Artificial sequence
  Synthetic
VVAA

SEQ ID NO: 5 - protein, 7 aa, Artificial sequence
  Synthetic
KKAAVVK

SEQ ID NO: 6 - protein, 9 aa, Artificial sequence
  Synthetic
VVVAAAKKK

SEQ ID NO: 7 - protein, 9 aa, Artificial sequence
  Synthetic
VVVAAAEEE

SEQ ID NO: 8 - protein, 25 aa, Artificial sequence
  Synthetic
VVVAAAEEEGKLTWQELYQLKYKGI

SEQ ID NO: 9 - protein, 26 aa, Artificial sequence
  Synthetic
VVVAAAEEEEGKLTWQELYQLKYKGI

SEQ ID NO: 10 - protein, 22 aa, Artificial sequence
  Synthetic
VVAAEEGKLTWQELYQLKYKGI

SEQ ID NO: 11 - protein, 24 aa, Artificial sequence
  Synthetic
VVAAEEEEGKLTWQELYQLKYKGI

SEQ ID NO: 12 - protein, 27 aa, Artificial sequence
  Synthetic
VVAAEEEEGGGGKLTWQELYQLKYKGI

SEQ ID NO: 13 - protein, 24 aa, Artificial sequence
  Synthetic
AAGGEEEEGKLTWQELYQLKYKGI

SEQ ID NO: 14 - protein, 25 aa, Artificial sequence
  Synthetic
VEVAAEEEEGKLTWQELYQLKYKGI

SEQ ID NO: 15 - protein, 23 aa, Artificial sequence
  Synthetic
VVAAKKKGKLTWQELYQLKYKGI

SEQ ID NO: 16 - protein, 26 aa, Artificial sequence
  Synthetic
VVAAKKKGGGGKLTWQELYQLKYKGI

SEQ ID NO: 17 - protein, 25 aa, Artificial sequence
  Synthetic
VVVAAAKKKGKLTWQELYQLKYKGI

(19) United States
(12) Patent Application Publication
(10) Pub. No.: US 2022/0118051 A1
(43) Pub. Date: Apr. 21, 2022

(54) SELF-ASSEMBLING VEGF NANOPARTICLES

(71) Applicant: Northwestern University, Evanston, IL (US)

(72) Inventors: Puneet Opal, Oak Park, IL (US); Samuel I. Stupp, Chicago, IL (US)

(21) Appl. No.: 17/292,265

(22) PCT Filed: Nov. 8, 2019
(86) PCT No.: PCT/US2019/060466

§ 371 Date: May 7, 2021

Related U.S. Application Data
(60) Provisional application No. 62/758,219, filed on Nov. 9, 2018.

(51) Int. Cl. A61K 38/18; A61K 9/14; A61P 25/28

(52) U.S. Cl. CPC A61K 38/1866; A61P 25/28; A61K 9/14

(57) ABSTRACT

Provided herein are self-assembling peptide amphiphiles (PAs) comprising a bioactive vascular endothelial growth factor (VEGF) peptide, nanofibers displaying VEGF PAs, and methods of treatment or prevention of the polyglutamine disease Spinocerebellar Ataxia Type 1 (SCA1) and other neurodegenerative diseases therewith. In particular embodiments, a VEGF peptide delivery platform is provided in which the mechanical properties of the nanofiber material are tunable by altering the ratio of bioactive PA to structural PAs (e.g., acidic or basic PAs lacking a bioactive epitope).

### CROSS REFERENCE TO RELATED APPLICATIONS

The present application claims priority to and the benefit of U.S. Provisional Patent Application No. 62/758,219, filed Nov. 9, 2018, which is incorporated herein by reference in its entirety and for all purposes.

### STATEMENT REGARDING FEDERAL FUNDING

This invention was made with government support under NS099962 and NS082351 awarded by the National Institutes of Health. The government has certain rights in the invention.

### FIELD

Provided herein are self-assembling peptide amphiphiles (PAs) comprising a bioactive vascular endothelial growth factor (VEGF) peptide, nanofibers displaying VEGF PAs, and methods of treatment or prevention of the polyglutamine disease Spinocerebellar Ataxia Type 1 (SCA1) and other neurodegenerative diseases therewith. In particular embodiments, a VEGF peptide delivery platform is provided in which the mechanical properties of the nanofiber material are tunable by altering the ratio of bioactive PA to structural PAs (e.g., acidic or basic PAs lacking a bioactive epitope).

### BACKGROUND

The leading neurodegenerative diseases—Alzheimer's, Parkinson's, amyotrophic lateral sclerosis, Huntington's disease and other polyglutaminopathies—are clinically and etiologically heterogeneous. Yet it is increasingly apparent that these diseases, long considered strictly in terms of neuronal dysfunction, also disrupt the close coupling of neural activity with blood flow (Iadecola, 2017; herein incorporated by reference in their entireties). Several years ago, it was discovered that the angiogenic and neurotrophic factor, VEGF (vascular endothelial growth factor) (Jin et al., 2002; Holmes and Zachary, 2005; herein incorporated by reference in their entireties), is downregulated in the polyglutamine disease Spinocerebellar Ataxia Type 1 (SCA1); in fact, the protein Ataxin-1 directly represses VEGF expression, and polyglutamine-expanded Ataxin-1 represses VEGF even more strongly (Cvetanovic et al., 2011; herein incorporated by reference in its entirety). SCA1 mice have very low levels of VEGF in the cerebellum, the part of the brain most vulnerable to the disease, and they show deficits in the cerebellar vasculature and dendritic arborization. Genetically or chemically replenishing VEGF mitigates the ataxia and dendritic defects of the SCA1 mice, raising the tantalizing possibility that SCA1 patients might benefit from VEGF treatment (Cvetanovic et al., 2011; herein incorporated by reference in its entirety). More recent studies in related proteopathies indicate that VEGF may also be therapeutic in Parkinson's disease (Caballero et al., 2017; Zou et al., 2017; herein incorporated by reference in their entireties), Alzheimer's disease (Echeverria et al., 2017; herein incorporated by reference in its entirety), and amyotrophic lateral sclerosis (Wang et al., 2016; herein incorporated by reference in its entirety).

Unfortunately, significant impediments prevent the translation of recombinant VEGF therapy to the clinic. Not only is it extremely costly to manufacture, but it is biologically unstable (prone to proteolysis) and has a half-life of only about 30 minutes in vivo (Thorne and Frey, 2001; Storkebaum et al., 2005; herein incorporated by reference in their entireties). It is also immunogenic, with antibodies directed against different epitopes interfering with its effects. A completely synthetic VEGF mimetic peptide was developed to overcome these issues, in which a fifteen-amino acid VEGF sequence (KLTWQELYQLKYKGI (SEQ ID NO: 1) is covalently linked to an amphiphilic peptide (Webber et al., 2011; herein incorporated by reference in its entirety). These amino acids mimic VEGF residues 17 through 25 in their ability to bind and activate VEGF receptors (D'Andrea et al., 2005; Webber et al., 2011; herein incorporated by reference in their entireties). In the aqueous tissue environment, the peptide backbone promotes intermolecular β-sheet hydrogen bonding, leading to self-assembly of the VEGF peptide molecules into cylindrical nanostructures that express VEGF-mimetic epitopes on the surface at a high van der Waal density (˜1015/cm2). The VEGF-mimetic peptide amphiphile (“VEGF-PA” or “nano-VEGF”), engages with VEGF receptors to result in potent signaling and is designed to eventually biodegrade. This peptide had been previously tested in a mouse hind-limb ischemia model, which demonstrated that the particles are retained in tissue even four weeks after delivery (Webber et al., 2011; herein incorporated by reference in its entirety) before slowly breaking apart (Silva et al., 2004; Webber et al., 2011; herein incorporated by reference in their entireties). Nano-VEGF had never been tested in the brain.

### SUMMARY

Provided herein are self-assembling peptide amphiphiles (PAs) comprising a bioactive vascular endothelial growth factor (VEGF) peptide, nanofibers displaying VEGF PAs, and methods of treatment or prevention of the polyglutamine disease Spinocerebellar Ataxia Type 1 (SCA1) and other neurodegenerative diseases therewith. In particular embodiments, a VEGF peptide delivery platform is provided in which the mechanical properties of the nanofiber material are tunable by altering the ratio of bioactive PA to structural PAs (e.g., acidic or basic PAs lacking a bioactive epitope).

In some embodiments, provided herein are peptide amphiphile nanofibers comprising: (a) a bioactive peptide amphiphile comprising: (i) a hydrophobic non-peptidic segment; (ii) a β-sheet-forming peptide segment; (iii) a charged peptide segment; and (iv) a VEGF peptide comprising at least 50% sequence identity with KLTWQELYQLKYKGI (SEQ ID NO:1); and (b) a charged peptide amphiphile comprising: (i) a hydrophobic non-peptidic segment; (ii) a β-sheet-forming peptide segment; and (iii) a charged peptide segment.

In some embodiments, the charged peptide amphiphile does not comprise a bioactive peptide.

In some embodiments, the hydrophobic non-peptidic segment of the bioactive peptide amphiphile and the charged peptide amphiphile comprises an acyl chain. In some embodiments, the acyl chain comprises C₆-C₂₀. In some embodiments, the acyl chain comprises C16.

In some embodiments, the β-sheet-forming peptide segment of the bioactive peptide amphiphile and the charged peptide amphiphile comprises AAAVVV (SEQ ID NO:2) or AAVV (SEQ ID NO:3).

In some embodiments, the charged peptide segment of the bioactive peptide amphiphile is an acidic peptide segment. In some embodiments, the acidic peptide segment of the bioactive peptide amphiphile comprises Glu (E) and/or Asp (D) residues. In some embodiments, the acidic peptide segment of the bioactive peptide amphiphile comprises is 2-7 amino acids in length with 50% or more amino acids selected from Glu (E) and/or Asp (D) residues. In some embodiments, the acidic peptide segment of the bioactive peptide amphiphile comprises EEE.

In some embodiments, the charged peptide segment of the bioactive peptide amphiphile is a basic peptide segment. In some embodiments, the basic peptide segment of the bioactive peptide amphiphile comprises one or more lysine (K), histidine (H), and/or arginine (R) residues. In some embodiments, the basic peptide segment of the bioactive peptide amphiphile comprises is 2-7 amino acids in length with 50% or more Lys (K) residues. In some embodiments, the basic peptide segment of the bioactive peptide amphiphile comprises KKK.

In some embodiments, the charged peptide segment of the charged peptide amphiphile is an acidic peptide segment. In some embodiments, the acidic peptide segment of the charged peptide amphiphile comprises Glu (E) and/or Asp (D) residues. In some embodiments, the acidic peptide segment of the charged peptide amphiphile comprises is 2-7 amino acids in length with 50% or more amino acids selected from Glu (E) and/or Asp (D) residues. In some embodiments, the acidic peptide segment of the charged peptide amphiphile comprises EEE.

In some embodiments, the charged peptide segment of the charged peptide amphiphile is a basic peptide segment. In some embodiments, the basic peptide segment of the charged peptide amphiphile comprises one or more lysine (K), histidine (H), and/or arginine (R) residues. In some embodiments, the basic peptide segment of the charged peptide amphiphile comprises is 2-7 amino acids in length with 50% or more Lys (K) residues. In some embodiments, the basic peptide segment of the charged peptide amphiphile comprises KKK.

In some embodiments, the VEGF peptide comprises at least 70% sequence identity with SEQ ID NO: 1. In some embodiments, the VEGF peptide comprises SEQ ID NO: 1.

In some embodiments, the peptide amphiphile nanofiber comprises 5%-75% by mass bioactive peptide amphiphile and 25% to 95% by mass basic peptide amphiphile, and wherein the nanofiber forms a gel under basic conditions.

In some embodiments, the peptide amphiphile nanofiber comprises 5%-75% (by mass) bioactive peptide amphiphile and 25% to 75% (by mass) basic peptide amphiphile, and wherein the nanofiber forms a gel under basic conditions. In some embodiments, 5%, 10%, 15%, 20%, 25%, 30%, 35%, 40%, 45%, 50%, 55%, 60%, 65%, 70%, 75%, or ranges therebetween bioactive peptide amphiphile. In some embodiments, 25%, 30%, 35%, 40%, 45%, 50%, 55%, 60%, 65%, 70%, 75, or ranges therebetween basic peptide amphiphile.

In some embodiments, the peptide amphiphile nanofiber comprises 75-99% (by mass) bioactive peptide amphiphile and 1% to 25% (by mass) basic peptide amphiphile, and wherein the nanofiber is a liquid under basic conditions. In some embodiments, 75%, 80%, 85%, 90%, 95%, 99%, or ranges therebetween bioactive peptide amphiphile. In some embodiments, 1%, 5%, 10%, 15%, 20%, 25%, or ranges therebetween basic peptide amphiphile.

In some embodiments, the peptide amphiphile nanofiber comprises 1-20% (by mass) bioactive peptide amphiphile and 80-99% (by mass) acidic peptide amphiphile, and wherein the nanofiber forms a gel under acidic conditions. In some embodiments, 1%, 5%, 10%, 15%, 20%, or ranges therebetween bioactive peptide amphiphile. In some embodiments, 80%, 85%, 90%, 95%, 99%, or ranges therebetween acidic peptide amphiphile.

In some embodiments, the peptide amphiphile nanofiber comprises 20-80% (by mass) bioactive peptide amphiphile and 20-80% (by mass) acidic peptide amphiphile, and wherein the nanofiber forms a gel under neutral conditions. In some embodiments, 20%, 25%, 30%, 35%, 40%, 45%, 50%, 55%, 60%, 65%, 70%, 75%, 80%, or ranges therebetween bioactive peptide amphiphile. In some embodiments, 20%, 25%, 30%, 35%, 40%, 45%, 50%, 55%, 60%, 65%, 70%, 75%, 80%, or ranges therebetween acidic peptide amphiphile.

In some embodiments, the peptide amphiphile nanofiber comprises 80-99% (by mass) bioactive peptide amphiphile and 1-20% (by mass) acidic peptide amphiphile, and wherein the nanofiber is a liquid under acidic conditions. In some embodiments, 80%, 85%, 90%, 95%, 99%, or ranges therebetween bioactive peptide amphiphile. In some embodiments, 1%, 5%, 10%, 15%, 20%, or ranges therebetween acidic peptide amphiphile.

In some embodiments, provided herein is a peptide amphiphile nanofiber comprising: (a) a bioactive peptide amphiphile comprising: (i) a hydrophobic non-peptidic segment comprising a C₆-C₂₀ acyl chain; (ii) a β-sheet-forming peptide segment comprising AAAVVV (SEQ ID NO: 2) or AAVV (SEQ ID NO: 3); (iii) a charged peptide segment, wherein the charged peptide segment comprises: (A) an acidic peptide segment comprising EEE, EED, EDE, DEE, EDD, DED, DDE, or DDD; or (B) a basic peptide segment comprising 2-7 or more lysine (K), histidine (H), and/or arginine (R) residues; and (iv) a VEGF peptide comprising at least 50% sequence identity with KLTWQELYQLKYKGI (SEQ ID NO:1); and (b) a charged peptide amphiphile comprising: (i) a hydrophobic non-peptidic segment comprising a C₆-C₂₀ acyl chain; (ii) a β-sheet-forming peptide segment comprising AAAVVV (SEQ ID NO: 2) or AAVV (SEQ ID NO: 3); and (iii) a charged peptide segment, wherein the charged peptide segment comprises: (A) an acidic peptide segment comprising EEE, EED, EDE, DEE, EDD, DED, DDE, or DDD; or (B) a basic peptide segment comprising 2-7 or more lysine (K), histidine (H), and/or arginine (R) residues.

In some embodiments, a peptide amphiphile herein comprises a linker segment between the charged peptide segment and the bioactive peptide segment. In some embodiments, the linker segment comprises 1-3 glycine (G) residues.

In some embodiments, a bioactive peptide amphiphile herein comprises with at least 70% sequence identity (e.g., 70%, 75%, 80%, 85%, 90%, 95%, or ranges therebetween) with one selected from one of C16-V3A3E3G-KLTWQELYQLKYKGI (SEQ ID NO: 8), C16-V3A3E4G-KLTWQELYQLKYKGI (SEQ ID NO: 9), C16-V2A2E2G-KLTWQELYQLKYKGI (SEQ ID NO: 10), C16-V2A2E4G-KLTWQELYQLKYKGI (SEQ ID NO: 11), C16-V2A2E4G4-KLTWQELYQLKYKGI (SEQ ID NO: 12), C16-A2G2E4G-KLTWQELYQLKYKGI (SEQ ID NO: 13), C16-VEVA2E4G-KLTWQELYQLKYKGI (SEQ ID NO: 14), C16-V2A2K3G-KLTWQELYQLKYKGI (SEQ ID NO: 15), C16-V2A2K3G4-KLTWQELYQLKYKGI (SEQ ID NO: 16), and C16-V3A3K3G-KLTWQELYQLKYKGI (SEQ ID NO: 17). In some embodiments, a bioactive peptide amphiphile selected from one of C16-V3A3E3G-KLTWQELYQLKYKGI (SEQ ID NO: 8), C16-V3A3E4G-KLTWQELYQLKYKGI (SEQ ID NO: 9), C16-V2A2E2G-KLTWQELYQLKYKGI (SEQ ID NO: 10), C16-V2A2E4G-KLTWQELYQLKYKGI (SEQ ID NO: 11), C16-V2A2E4G4-KLTWQELYQLKYKGI (SEQ ID NO: 12), C16-A2G2E4G-KLTWQELYQLKYKGI (SEQ ID NO: 13), C16-VEVA2E4G-KLTWQELYQLKYKGI (SEQ ID NO: 14), C16-V2A2K3G-KLTWQELYQLKYKGI (SEQ ID NO: 15), C16-V2A2K3G4-KLTWQELYQLKYKGI (SEQ ID NO: 16), and C16-V3A3K3G-KLTWQELYQLKYKGI (SEQ ID NO: 17).

In some embodiments, provided herein are methods of treating a neurologic condition comprising administering a pharmaceutical composition comprising a peptide amphiphile nanofiber described herein to a subject suffering from the neurologic condition. In some embodiments, the neurologic condition is a polyglutamine disease. In some embodiments, the polyglutamine disease Spinocerebellar Ataxia Type 1. In some embodiments, the pharmaceutical composition is administered parenterally. In some embodiments, the pharmaceutical composition is administered by intrathecal administration, intracerebroventricular administration, or intraparenchymal administration.

### BRIEF DESCRIPTION OF THE DRAWINGS

FIG. 1A-D. Nano-VEGF delivery improves vascular physiology in SCA1 mice. (A) Timeline of treatment and subsequent behavioral and pathological analyses. (B) Nano-VEGF and rVEGF both improved rotarod performance of SCA1 mice at 26 weeks (compared to SCA1 mice treated with aCSF), with nano-VEGF being more effective. Data represent mean±SE. Two-way ANOVA follow by Tukey's test. *p<0.05, ***p<0.005. (C) Nano-VEGF and rVEGF treatment significantly increase dendritic length and improves morphology of Purkinje cells in SCA1 mice as assayed by calbindin staining (scale bar=100 μm). This is quantified in D. (D) Data represent mean±SE. At least 3 mice at 27 weeks were analyzed in each experimental group. Statistical analysis: One-way ANOVA followed by the Tukey's post hoc test. ***p<0.005, ****p<0.0001.

FIG. 2A-H. Nano-VEGF significantly rescues cerebellar motor phenotype in late symptomatic SCA1 mice with effects comparable to or better than recombinant VEGF (A) ELISA shows phosphorylated VEGFR2 levels are increased in VEGF-treated SCA1 mice compared to aCSF-treated controls. N=5-6 mice. (B) Immunostaining of Collagen IV marker for microvessels in different treatment groups (scale bar=100 μm). (C) VEGF treatment increases average vessel length and (D) branching index (N=3 per treatment group). (E) SCA1 mice display abnormally low levels of VEGF and tight junction proteins in the cerebella, but VEGF treatment increases the level of junction proteins in these mice. (F-H) Quantifications of western blot expression level of ZO-1, Claudin-5 and Occludin normalized to actin level. All values are mean±SEM., one-way analysis of variance (ANOVA), followed by Tukey's post hoc test. *P<0.05; ** P<0.001.

FIG. 3A-D. Nano-VEGF treatment does not alter Ataxin-1 levels in SCA1 mice. (A) Western blot of Ataxin-1 mutant and wildtype shows protein levels were not affected by two weeks of VEGF treatment. (B) Immunohistochemistry of Ataxin-1 inclusions in the hippocampi of nano-VEGF and aCSF treated SCA1 mice (scale bar=150 μm). Inserts show 63× magnification images of Ataxin-1 inclusion in CA1 region (scale bar=10 μm), one-way analysis of variance (ANOVA), followed by Tukey's post hoc test. (C) Percentage of cells with Ataxin-1 inclusions in the CA1 of hippocampi of nano-VEGF treated mice does not differ from that of aCSF-treated mice, suggesting that VEGF treatment does not alter clearance of Ataxin-1 aggregates. (D) Quantification of aggregates, unpaired t-test.

FIG. 4A-C. Nano-VEGF treatment increases firing in Purkinje cells from SCA1 mice. (A) Representative cell-attached recordings of spontaneous firing in Purkinje cells of 12-week-old mice treated with either vehicle (aCSF) or nano-VEGF. The PCs from VEGF treated mice (red trace) fired at higher frequencies than vehicle-treated mice (black trace). (B) Representative probability distribution of inter-spike intervals in PC from vehicle-treated and VEGF-treated mice. (C) Box plot recapitulates the grouped difference in PC firing frequency in the two experimental conditions (vehicle N=10, VEGF N=8, unpaired t-test, **p=0.012).

FIG. 5. VEGF levels are suppressed in SCA1 patients. (A) Representative images (10× magnification) of cerebella from SCA1 patients and controls stained for VEGF; scale bar=100 μm. Inset shows a higher magnification (40×) of the Purkinje cell layer; scale bar=50 μm. (B) Quantification of VEGF intensity of SCA1 patient cerebella (N=5) is compared to healthy controls (N=4). The Optical Density was measured for the entire image using color-deconvolution system that can separate the contribution of up to three stains to the final color subtracted. Data represent mean±SE. *P<0.05; Student's t test.

FIG. 6A-C. VEGF effects in wild-type mice. (A) Rotarod data for 26-week-old male WT mice treated with aCSF and VEGF shows overall no significant difference between the two groups over time. (B) VEGF treatment of wild-type mice lengthened PC dendrites. (C) rVEGF and nano-VEGF did not upregulate phosphorylation of VEGFR2 in WT mice, suggesting that there is a ceiling effect.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Om”), pentylglycine (“pG” or “PGly”), pipecolic acid, thioproline (“ThioP” or “tPro”), homoLysine (“hLys”), and homoArginine (“hArg”).

The term “amino acid analog” refers to a natural or unnatural amino acid where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “peptide” refers an oligomer to short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (artificial) sequence.

As used herein, the term “artificial” refers to compositions and systems that are designed or prepared by man, and are not naturally occurring. For example, an artificial peptide, peptoid, or nucleic acid is one comprising a non-natural sequence (e.g., a peptide without 100% identity with a naturally-occurring protein or a fragment thereof).

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Naturally occurring residues may be divided into classes based on common side chain properties, for example: polar positive (or basic) (histidine (H), lysine (K), and arginine (R)); polar negative (or acidic) (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree of which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any polypeptides described herein as having a particular percent sequence identity or similarity (e.g., at least 70%) with a reference sequence ID number, may also be expressed as having a maximum number of substitutions (or terminal deletions) with respect to that reference sequence. For example, a sequence having at least Y % sequence identity (e.g., 90%) with SEQ ID NO:Z (e.g., 100 amino acids) may have up to X substitutions (e.g., 10) relative to SEQ ID NO:Z, and may therefore also be expressed as “having X (e.g., 10) or fewer substitutions relative to SEQ ID NO:Z.”

As used herein, the term “nanofiber” refers to an elongated or threadlike filament (e.g., having a significantly greater length dimension that width or diameter) with a diameter typically less than 100 nanometers.

As used herein, the term “supramolecular” (e.g., “supramolecular complex,” “supramolecular interactions,” “supramolecular fiber,” “supramolecular polymer,” etc.) refers to the non-covalent interactions between molecules (e.g., polymers, macromolecules, etc.) and the multicomponent assemblies, complexes, systems, and/or fibers that form as a result.

As used herein, the terms “self-assemble” and “self-assembly” refer to formation of a discrete, non-random, aggregate structure from component parts; said assembly occurring spontaneously through random movements of the components (e.g. molecules) due only to the inherent chemical or structural properties and attractive forces of those components.

As used herein, the term “peptide amphiphile” refers to a molecule that, at a minimum, includes a non-peptide lipophilic (hydrophobic) segment, a structural peptide segment and/or charged peptide segment (often both), and optionally a bioactive segment (e.g., linker segment, bioactive segment, etc.). The peptide amphiphile may express a net charge at physiological pH, either a net positive or negative net charge, or may be zwitterionic (i.e., carrying both positive and negative charges). Certain peptide amphiphiles consist of or comprise: (1) a hydrophobic, non-peptide segment (e.g., comprising an acyl group of six or more carbons), (2) a structural peptide segment (e.g., β-sheet forming); (3) a charged peptide segment, and (4) a bioactive segment (e.g., linker segment).

As used herein and in the appended claims, the term “lipophilic moiety” or “hydrophobic moiety” refers to the moiety (e.g., an acyl, ether, sulfonamide, or phosphodiester moiety) disposed on one terminus (e.g., C-terminus, N-terminus) of the peptide amphiphile, and may be herein and elsewhere referred to as the lipophilic or hydrophobic segment or component. The hydrophobic segment should be of a sufficient length to provide amphiphilic behavior and aggregate (or nanosphere or nanofiber) formation in water or another polar solvent system. Accordingly, in the context of the embodiments described herein, the hydrophobic component preferably comprises a single, linear acyl chain of the formula: Cₙ₋₁H₂ₙ₋₁C(O)— where n=2-25. In some embodiments, a linear acyl chain is the lipophilic group (saturated or unsaturated carbons), palmitic acid. However, other lipophilic groups may be used in place of the acyl chain such as steroids, phospholipids and fluorocarbons.

As used herein, the term “structural peptide” refers to a portion of a peptide amphiphile, typically disposed between the hydrophobic segment and the charged peptide segment. The structural peptide is generally composed of three to ten amino acid residues with non-polar, uncharged side chains (e.g., His (H), Val (V), Ile (I), Leu (L), Ala (A), Phe (F)) selected for their propensity to form hydrogen bonds or other stabilizing interactions (e.g., hydrophobic interactions, van der Waals' interactions, etc.) with structural segments of adjacent structural segments. In some embodiments, nanofibers of peptide amphiphiles having structural peptide segments display linear or 2D structure when examined by microscopy and/or α-helix and/or β-sheet character when examined by circular dichroism (CD).

As used herein, the term “beta (β)-sheet-forming peptide segment” refers to a structural peptide segment that has a propensity to display β-sheet-like character (e.g., when analyzed by CD). In some embodiments, amino acids in a beta (β)-sheet-forming peptide segment are selected for their propensity to form a beta-sheet secondary structure. Examples of suitable amino acid residues selected from the twenty naturally occurring amino acids include Met (M), Val (V), Ile (I), Cys (C), Tyr (Y), Phe (F), Gln (Q), Leu (L), Thr (T), Ala (A), and Gly (G) (listed in order of their propensity to form beta sheets). However, non-naturally occurring amino acids of similar beta-sheet forming propensity may also be used. Peptide segments capable of interacting to form beta sheets and/or with a propensity to form beta sheets are understood (See, e.g., Mayo et al. Protein Science (1996), 5:1301-1315; herein incorporated by reference in its entirety).

As used herein, the term “charged peptide segment” refers to a portion of a peptide amphiphile that is rich (e.g., >50%, >75%, etc.) in charged amino acid residues, or amino acid residue that have a net positive or negative charge under physiologic conditions. A charged peptide segment may be acidic (e.g., negatively charged), basic (e.g., positively charged), or zwitterionic (e.g., having both acidic and basic residues).

As used herein, the terms “carboxy-rich peptide segment,” “acidic peptide segment,” and “negatively-charged peptide segment” refer to a peptide sequence of a peptide amphiphile that comprises one or more amino acid residues that have side chains displaying carboxylic acid side chains (e.g., Glu (E), Asp (D), or non-natural amino acids). A carboxy-rich peptide segment may optionally contain one or more additional (e.g., non-acidic) amino acid residues. Non-natural amino acid residues, or peptidomimetics with acidic side chains could be used, as will be evident to one ordinarily skilled in the art. There may be from about 2 to about 7 amino acids, and or about 3 or 4 amino acids in this segment.

As used herein, the terms “amino-rich peptide segment”, “basic peptide segment,” and “positively-charged peptide segment” refer to a peptide sequence of a peptide amphiphile that comprises one or more amino acid residues that have side chains displaying positively-charged acid side chains (e.g., Arg (R), Lys (K), His (H), or non-natural amino acids, or peptidomimetics). A basic peptide segment may optionally contain one or more additional (e.g., non-basic) amino acid residues. Non-natural amino acid residues with basic side chains could be used, as will be evident to one ordinarily skilled in the art. There may be from about 2 to about 7 amino acids, and or about 3 or 4 amino acids in this segment.

As used herein, the term “bioactive peptide” refers to amino acid sequences that mediate the action of sequences, molecules, or supramolecular complexes associated therewith. Peptide amphiphiles and structures (e.g., nanofibers) bearing bioactive peptides (e.g., a TF-targeting sequence, etc.) exhibits the functionality of the bioactive peptide.

As used herein, the term “biocompatible” refers to materials and agents that are not toxic to cells or organisms. In some embodiments, a substance is considered to be “biocompatible” if its addition to cells in vitro results in less than or equal to approximately 10% cell death, usually less than 5%, more usually less than 1%.

As used herein, “biodegradable” as used to describe the polymers, hydrogels, and/or wound dressings herein refers to compositions degraded or otherwise “broken down” under exposure to physiological conditions. In some embodiments, a biodegradable substance is a broken down by cellular machinery, enzymatic degradation, chemical processes, hydrolysis, etc. In some embodiments, a wound dressing or coating comprises hydrolyzable ester linkages that provide the biodegradability.

As used herein, the phrase “physiological conditions” relates to the range of chemical (e.g., pH, ionic strength) and biochemical (e.g., enzyme concentrations) conditions likely to be encountered in the intracellular and extracellular fluids of tissues. For most tissues, the physiological pH ranges from about 7.0 to 7.4.

As used herein, the terms “treat,” “treatment,” and “treating” refer to reducing the amount or severity of a particular condition, disease state (e.g., SCA1), or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

As used herein, the terms “prevent,” “prevention,” and preventing” refer to reducing the likelihood of a particular condition or disease state (e.g., SCA1) from occurring in a subject not presently experiencing or afflicted with the condition or disease state. The terms do not necessarily indicate complete or absolute prevention. For example “preventing SCA1” refers to reducing the likelihood of SCA1 occurring in a subject not presently experiencing or diagnosed with SCA1. In order to “prevent SCA1” a composition or method need only reduce the likelihood of SCA1, not completely block any possibility thereof. “Prevention,” encompasses any administration or application of a therapeutic or technique to reduce the likelihood of a disease developing (e.g., in a mammal, including a human). Such a likelihood may be assessed for a population or for an individual.

As used herein, the terms “co-administration” and “co-administering” refer to the administration of at least two agent(s) or therapies to a subject (e.g., a VEGF PA nanofiber and one or more therapeutic agents). In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

### DETAILED DESCRIPTION

Provided herein are self-assembling peptide amphiphiles (PAs) comprising a bioactive vascular endothelial growth factor (VEGF) peptide, nanofibers displaying VEGF PAs, and methods of treatment or prevention of the polyglutamine disease Spinocerebellar Ataxia Type 1 (SCA1) and other neurodegenerative diseases therewith. In particular embodiments, a VEGF peptide delivery platform is provided in which the mechanical properties of the nanofiber material are tunable by altering the ratio of bioactive PA to structural PAs (e.g., acidic or basic PAs lacking a bioactive epitope).

Experiments were conducted during development of embodiments herein to test a synthetic nano-VEGF construct, with a functional epitope designed to mimic the N-terminal helical domain of VEGF (residues 17-25) (Webber et al., 2011; herein incorporated by reference in its entirety). Nano-VEGF activates the VEGF receptor (VEGFR2) and improves the SCA1 phenotype on both behavioral and pathological assays. Nano-VEGF outperformed recombinant VEGF in several respects, such as improving the levels of capillary proteins and microvascular health. As a pharmacological agent, nano-VEGF has several advantages over recombinant VEGF. Full-length recombinant VEGF is difficult to synthesize because of the protein's complex structure: it is a glycoprotein formed from two 165-amino acid monomers that must be joined by three interconnected disulfide bridges in a cysteine knot motif. Generating functional VEGF from bacterial and other expression systems is not only costly but inefficient, with considerable variations from one batch to the next (Storkebaum et al., 2005; herein incorporated by reference in its entirety). Beyond these practical limitations, recombinant VEGF has poor pharmacokinetics, with a very short half-life (Thome and Frey, 2001; Storkebaum et al., 2005; herein incorporated by reference in their entireties). Nano-VEGF, on the other hand, is stable (detectable in an ischemic limb model up to 4 weeks post-injection) and forms small filamentous structures that break apart slowly, providing a slow-release formulation. In addition, the polyvalent nature of VEGF mimetic peptides on the surface is thought to promote receptor dimerization and sustained activation (Webber et al., 2011; herein incorporated by reference in its entirety). These parameters, contribute to nano-VEGF performing so favorably compared to recombinant VEGF.

The VEGF peptide itself is designed to engage with VEGFR1 and 2 (Wiesmann et al., 1997; Diana et al., 2008; herein incorporated by reference in their entireties). While it is not the only region of VEGF that has been shown to activate these receptors, this peptide has been the best characterized in functional models. It is contemplated that two other domains (VEGF residues 61-66 and residues 79-93) also engage VEGF receptors. In some embodiments, provided herein are peptide amphiphiles incorporating these sequences with that of the nano-VEGF peptide tested herein, and/or nanofibers comprising PAs displaying different VEGF peptides (e.g., 17-25, 61-66, and/or 79-93).

Intracerebroventricular (ICV) delivery of nano-VEGF was conducted to avoid systemic effects (e.g., promoting tumorigenesis (Carmeliet, 2005; herein incorporated by reference in its entirety), hypotension (Yang et al., 2002; herein incorporated by reference in its entirety), or coagulation disorders (Verheul et al., 2010; herein incorporated by reference in its entirety)). There are alternatives to ICV, such as intraparenchymal delivery, that may also find use in embodiments herein. Since VEGF by ICV enters the cerebrospinal fluid and bathes the brain, all circuits and cell types are being targeted. Furthermore, FDA-approved battery-operated pumps that deliver drugs via a subcutaneously placed catheter into the CSF for long-term drug delivery are available, which obviate the need for repeated intraparenchymal injections (Bottros and Christo, 2014; herein incorporated by reference in its entirety).

VEGF levels are abnormally suppressed in several neurodegenerative diseases, including ALS, spinobulbar muscular atrophy (SBMA), Parkinson's, and Alzheimer's disease. In some embodiments, nano-VEGF is provided for the treatment/prevention/symptom reduction in any of these diseases. Experiments conducted during development of embodiments herein indicate that self-assembling nanoparticles provide an effective therapeutic platform for delivery of treatment for a variety of neurodegenerative disorders.

In some embodiments, the peptide amphiphile molecules and compositions of the embodiments described herein are synthesized using preparatory techniques well-known to those skilled in the art, preferably, by standard solid-phase peptide synthesis, with the addition of a fatty acid in place of a standard amino acid at the N-terminus (or C-terminus) of the peptide, in order to create the lipophilic segment (although in some embodiments, alignment of nanofibers is performed via techniques not previously disclosed or used in the art (e.g., extrusion through a mesh screen). Synthesis typically starts from the C-terminus, to which amino acids are sequentially added using either a Rink amide resin (resulting in an —NH2 group at the C-terminus of the peptide after cleavage from the resin), or a Wang resin (resulting in an —OH group at the C-terminus). Accordingly, some embodiments described herein encompass peptide amphiphiles having a C-terminal moiety that may be selected from the group consisting of —H, —OH, —COOH, —CONH2, and —NH2.

In some embodiments, peptide amphiphiles comprise a hydrophobic (non-peptide) segment linked to a peptide. In some embodiments, the peptide comprises a structural segment (e.g., hydrogen-bond-forming segment, beta-sheet-forming segment, etc.), and a charged segment (e.g., acidic segment, basic segment, zwitterionic segment, etc.). In some embodiments, the peptide further comprises linker or spacer segments for adding solubility, flexibility, distance between segments, etc. In some embodiments, peptide amphiphiles comprise a spacer segment (e.g., peptide and/or non-peptide spacer) at the opposite terminus of the peptide from the hydrophobic segment. In some embodiments, the spacer segment comprises peptide and/or non-peptide elements. In some embodiments, the spacer segment comprises one or more bioactive groups (e.g., alkene, alkyne, azide, thiol, etc.). In some embodiments, various segments may be connected by linker segments (e.g., peptide (e.g., GG) or non-peptide (e.g., alkyl, OEG, PEG, etc.) linkers).

The lipophilic or hydrophobic segment is typically incorporated at the N- or C-terminus of the peptide after the last amino acid coupling, and is composed of a fatty acid or other acid that is linked to the N- or C-terminal amino acid through an acyl bond. In aqueous solutions, PA molecules self-assemble (e.g., into cylindrical micelles (a.k.a., nanofibers)) that bury the lipophilic segment in their core and display the bioactive peptide on the surface. The structural peptide undergoes intermolecular hydrogen bonding to form beta sheets that orient parallel to the long axis of the micelle.

In some embodiments, compositions described herein comprise PA building blocks that in turn comprise a hydrophobic segment and a peptide segment. In certain embodiments, a hydrophobic (e.g., hydrocarbon and/or alkyl/alkenyl/alkynyl tail, or steroid such as cholesterol) segment of sufficient length (e.g., 2 carbons, 3 carbons, 4 carbons, 5 carbons, 6 carbons, 7 carbons, 8 carbons, 9 carbons, 10 carbons, 11 carbons, 12 carbons, 13 carbons, 14 carbons, 15 carbons, 16 carbons, 17 carbons, 18 carbons, 19 carbons, 20 carbons, 21 carbons, 22 carbons, 23 carbons, 24 carbons, 25 carbons, 26 carbons, 27 carbons, 28 carbons, 29 carbons, 30 carbons or more, or any ranges there between.) is covalently coupled to peptide segment (e.g., a peptide comprising a segment having a preference for beta-strand conformations or other supramolecular interactions) to yield a peptide amphiphile molecule. In some embodiments, a plurality of such PAs will self-assemble in water (or aqueous solution) into a nanostructure (e.g., nanofiber). In various embodiments, the relative lengths of the peptide segment and hydrophobic segment result in differing PA molecular shape and nanostructural architecture. For example, a broader peptide segment and narrower hydrophobic segment results in a generally conical molecular shape that has an effect on the assembly of PAs (See, e.g., J. N. Israelachvili Intermolecular and surface forces; 2nd ed.; Academic: London San Diego, 1992; herein incorporated by reference in its entirety). Other molecular shapes have similar effects on assembly and nanostructural architecture.

In some embodiments, to induce self-assembly of an aqueous solution of peptide amphiphiles, the pH of the solution may be changed (raised or lowered) or multivalent ions, such as calcium, or charged polymers or other macromolecules may be added to the solution.

In some embodiments, the hydrophobic segment is a non-peptide segment (e.g., alkyl/alkenyl/alkynyl group). In some embodiments, the hydrophobic segment comprises an alkyl chain (e.g., saturated) of 4-25 carbons (e.g., 4, 5, 6, 7, 8, 9, 10, 11, 12, 13, 14, 15, 16, 17, 18, 19, 20, 21, 22, 23, 24, 25), fluorinated segments, fluorinated alkyl tails, heterocyclic rings, aromatic segments, pi-conjugated segments, cycloalkyls, oligothiophenes etc. In some embodiments, the hydrophobic segment comprises an acyl/ether chain (e.g., saturated) of 2-30 carbons (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30).

In some embodiments, PAs comprise one or more peptide segments. Peptide segment may comprise natural amino acids, modified amino acids, unnatural amino acids, amino acid analogs, peptidomimetics, or combinations thereof. In some embodiments, peptide segment comprise at least 50% sequence identity or similarity (e.g., conservative or semi-conservative) to one or more of the peptide sequences described herein.

In some embodiments, peptide amphiphiles comprise a charged peptide segment. The charged segment may be acidic, basic, or zwitterionic.

In some embodiments, peptide amphiphiles comprise an acidic peptide segment. For example, in some embodiments, the acidic peptide comprises one or more (e.g., 1, 2, 3, 4, 5, 6, 7, or more) acidic residues (D and/or E) in sequence. In some embodiments, the acidic peptide segment comprises up to 7 residues in length and comprises at least 50% acidic residues. In some embodiments, an acidic peptide segment comprises (Xa)₁₋₇, wherein each Xa is independently D or E. In some embodiments, an acidic peptide segment comprises EE.

In some embodiments, peptide amphiphiles comprise a basic peptide segment. For example, in some embodiments, the acidic peptide comprises one or more (e.g., 1, 2, 3, 4, 5, 6, 7, or more) basic residues (R, H, and/or K) in sequence. In some embodiments, the basic peptide segment comprises up to 7 residues in length and comprises at least 50% basic residues. In some embodiments, an acidic peptide segment comprises (Xb)₁₋₇, wherein each Xb is independently R, H, and/or K.

In some embodiments, peptide amphiphiles comprises a structural and/or beta-sheet-forming segment. In some embodiments, the structural segment is rich in H, I, L, F, V, and A residues. In some embodiments, the structural and/or beta-sheet-forming segment comprises an alanine- and valine-rich peptide segment (e.g., AAVV (SEQ ID NO: 3), AAAVVV (SEQ ID NO:2), or other combinations of V and A residues, etc.). In some embodiments, the structural and/or beta sheet peptide comprises 4 or more consecutive A and/or V residues, or conservative or semi-conservative substitutions thereto. In some embodiments, the structural and/or beta-sheet forming peptide segment comprises 4 or more consecutive non-polar aliphatic residues (e.g., alanine (A), valine (V), leucine (L), isoleucine (I), methionine (M)). In some embodiments, the structural and/or beta-sheet forming peptide segment comprises 2-16 amino acids in length and comprises 4 or more (e.g., 2, 3, 4, 5, 6, 7, 8, 9, 10, 11, 12, 13, 14, 15, 16, or ranges there between) non-polar aliphatic residues.

In some embodiments, peptide amphiphiles comprise a non-peptide spacer or linker segment. In some embodiments, the non-peptide spacer or linker segment is located at the opposite terminus of the peptide from the hydrophobic segment. In some embodiments, the spacer or linker segment provides the attachment site for a bioactive group. In some embodiments, the spacer or linker segment provides a reactive group (e.g., alkene, alkyne, azide, thiol, maleimide etc.) for functionalization of the PA. In some embodiments, the spacer or linker is a substantially linear chain of CH₂, O, (CH₂)₂O, O(CH₂)₂, NH, and C═O groups (e.g., CH₂(O(CH₂)₂)₂NH, CH₂(O(CH₂)₂)₂NHCO(CH₂)₂CCH, etc.). In some embodiments, a spacer or linker further comprises additional bioactive groups, substituents, branches, etc.

Suitable peptide amphiphiles for use in the materials herein, as well as methods of preparation of PAs and related materials, amino acid sequences for use in PAs, and materials that find use with PAs, are described in the following patents: U.S. Pat. Nos. 9,044,514; 9,040,626; 9,011,914; 8,772,228; 8,748,569 8,580,923; 8,546,338; 8,512,693; 8,450,271; 8,236,800; 8,138,140; 8,124,583; 8,114,835; 8,114,834; 8,080,262; 8,076,295; 8,063,014; 7,851,445; 7,838,491; 7,745,708; 7,683,025; 7,554,021; 7,544,661; 7,534,761; 7,491,690; 7,452,679; 7,371,719; 7,030,167; all of which are herein incorporated by reference in their entireties.

The characteristics (e.g., shape, rigidity, hydrophilicity, etc.) of a PA supramolecular structure depend upon the identity of the components of a peptide amphiphile (e.g., lipophilic segment, acidic segment, structural segment, bioactive segment, etc.). For example, nanofibers, nanospheres, intermediate shapes, and other supramolecular structures are achieved by adjusting the identity of the PA component parts. In some embodiments, characteristics of supramolecular nanostructures of PAs are altered by post-assembly manipulation (e.g., heating/cooling, stretching, etc.).

In some embodiments, a peptide amphiphile comprises: (a) a hydrophobic tail comprising an alkyl chain of 8-24 carbons; (b) a structural segment (e.g., comprising VVAA (SEQ ID NO: 4)); and (c) a charged segment (e.g., comprising KK, EE, etc.). In some embodiments, any PAs within the scope described herein, comprising the components described herein, or within the skill of one in the field, may find use herein.

In some embodiments, peptide amphiphiles comprise a bioactive moiety (e.g., VEGF peptide). In particular embodiments, a bioactive moiety is the most C-terminal or N-terminal segment of the PA. In some embodiments, the bioactive moiety is attached to the end of the charged segment. In some embodiments, the bioactive moiety is exposed on the surface of an assembled PA structure (e.g., nanofiber). A bioactive moiety is typically a peptide (e.g., gro VEGF peptide, etc.), but is not limited thereto. In some embodiments, a bioactive moiety is a peptide sequence that binds a peptide or polypeptide of interests, for example, a growth factor. In some embodiments, a VEGF peptide is provided as a PA bioactive moiety. In some embodiments, such VEGF peptide comprise at least 70% (e.g., 70%, 75%, 80%, 85%, 90%, 95%, 98%, 99%, or ranges therebetween) sequence identity with SEQ ID NO: 1. In some embodiments, a VEGF peptide is SEQ ID NO: 1. In some embodiments, nanofibers are provided comprising bioactive PAs displaying one or more of a peptide comprising at least 70% (e.g., 70%, 75%, 80%, 85%, 90%, 95%, 98%, 99%, or ranges therebetween) sequence identity with one of SEQ ID NO: 1; a peptide comprising at least 70% (e.g., 70%, 75%, 80%, 85%, 90%, 95%, 98%, 99%, or ranges therebetween) sequence identity with residues 61-66 of VEGF; and/or a peptide comprising at least 70% (e.g., 70%, 75%, 80%, 85%, 90%, 95%, 98%, 99%, or ranges therebetween) sequence identity with residues 79-93 of VEGF. In some embodiments, a bioactive peptide comprises conservative or semi-conservative substitutions relative to one of SEQ ID NO: 1, residues 61-66 of VEGF, and/or residues 79-93 of VEGF.

In some embodiments, a peptide amphiphile comprises: (a) a hydrophobic tail comprising an alkyl chain of 8-24 carbons; (b) a structural segment (e.g., comprising VVAA (SEQ ID NO: 4), AAVV (SEQ ID NO: 3), VA, AV, etc.); (c) a charged segment (e.g., comprising KK, EE, EK, KE, etc.), and a bioactive peptide (e.g., VEGF peptide). In some embodiments, a PA further comprises an attachment segment or residue (e.g., K) for attachment of the hydrophobic tail to the peptide portion of the PA. In some embodiments, the hydrophobic tail is attached to a lysine side chain.

In some embodiments, a peptide amphiphile comprises (e.g., from C-terminus to N-terminus or from N-terminus to C-terminus): bioactive peptide (e.g., VEGF peptide)-charged segment (e.g., comprising KK, EE, EK, KE, etc.)—structural segment (e.g., comprising VVAA (SEQ ID NO: 4), AAVV (SEQ ID NO: 3), VA, AV, etc.)—hydrophobic tail (e.g., comprising an alkyl chain of 8-24 carbons).

In some embodiments, a peptide amphiphile comprises (e.g., from C-terminus to N-terminus or from N-terminus to C-terminus): bioactive peptide (e.g., VEGF peptide)-charged segment (e.g., comprising KK, EE, EK, KE, etc.)—structural segment (e.g., comprising VVAA (SEQ ID NO: 4), AAVV (SEQ ID NO: 3), AV, etc.)—attachment segment or peptide (e.g., K)—hydrophobic tail (e.g., comprising an alkyl chain of 8-24 carbons).

In some embodiments, a peptide amphiphile comprises (e.g., from C-terminus to N-terminus or from N-terminus to C-terminus): bioactive peptide (e.g., growth factor or GF-targeting peptide)—KKAAVV(K) (SEQ ID NO: 5)—hydrophobic tail (e.g., comprising an alkyl chain of 8-24 carbons). In some embodiments, the hydrophobic tail is attached to the (K) sidechain.

In some embodiments, provided herein are nanofibers and nanostructures assembled from the peptide amphiphiles described herein. In some embodiments, a nanofiber is prepared by the self-assembly of the PAs described herein. In some embodiments, a nanofiber comprises or consists of PAs displaying a VEGF peptide. In some embodiments, the VEGF peptides are displayed on the surface of the nanofiber. In some embodiments, in addition to PAs displaying VEGF peptides, filler PAs are included in the nanofibers. In some embodiments, filler PAs are peptide amphiphiles, as described herein (e.g., structural segment, charged segment, hydrophobic segment, etc.), but lacking a bioactive moiety. In some embodiments, filler peptides are basic or acidic peptides lacking a bioactive moiety (e.g., V3A3K3, V3A3E3, etc.). In some embodiments, the filler PAs and VEGF PAs self-assemble into a nanofiber comprising both types of PAs. In some embodiments, nanostructures (e.g., nanofibers) assembled from the peptide amphiphiles described herein are provided.

In some embodiments, filler peptides (e.g., basic peptide, acidic peptides, etc.) impart mechanical characteristics to a material comprising the PA nanofibers described herein. In some embodiments, a nanofiber assembled from 0-75% (mass %) bioactive VEGF PA and 25-100% (mass %) basic filler PA (e.g., C16-VVVAAAKKK (SEQ ID NO: 6)) becomes a gel at basic pH conditions (e.g., pH 8.5-11). In some embodiments, a nanofiber assembled from 75-100% (mass %) bioactive VEGF PA and 0-25% (mass %) basic filler PA (e.g., C16-VVVAAAKKK (SEQ ID NO: 6)) is a liquid at basic pH conditions (e.g., pH 8.5-11). In some embodiments, a nanofiber assembled from 0-20% (mass %) bioactive VEGF PA and 80-100% (mass %) acidic filler PA (e.g., C₁₆-VVVAAAEEE (SEQ ID NO: 7)) becomes a gel at acidic pH conditions (e.g., pH 1-5). In some embodiments, a nanofiber assembled from 20-80% (mass %) bioactive VEGF PA and 20-80% (mass %) acidic filler PA (e.g., C₁₆-VVVAAAEEE (SEQ ID NO: 7)) becomes a gel at neutral pH conditions (e.g., pH 5-8.5). In some embodiments, a nanofiber assembled from 80-100% (mass %) bioactive VEGF PA and 0-20% (mass %) acidic filler PA (e.g., C16-VVVAAAEEE (SEQ ID NO: 7)) is a liquid at acidic pH conditions (e.g., pH 1-5).

In some embodiments, nanostructures are assembled from (1) PAs bearing a bioactive moiety (e.g., VEGF peptide) and (2) filler PAs (e.g., acidic or basic PAs not-labeled or not displaying a bioactive moiety, etc.). In some embodiments, nanostructures (e.g., nanofibers) comprise 95%, 90%, 85%, 80%, 75%, 70%, 65%, 60%, 55%, 50% 45%, 40%, 35%, 30%, 25%, 20%, 15%, 10%, 5%, 4%, 3%, 2%, or 1% (or any ranges there between) VEGF PAs. In some embodiments, nanostructures (e.g., nanofibers) comprise 95%, 90%, 85%, 80%, 75%, 70%, 65%, 60%, 55%, 50% 45%, 40%, 35%, 30%, 25%, 20%, 15%, 10%, 5%, 4%, 3%, 2%, or 1% (or any ranges there between) acidic filler PAs. In some embodiments, nanostructures (e.g., nanofibers) comprise 95%, 90%, 85%, 80%, 75%, 70%, 65%, 60%, 55%, 50% 45%, 40%, 35%, 30%, 25%, 20%, 15%, 10%, 5%, 4%, 3%, 2%, or 1% (or any ranges there between) basic filler PAs. In some embodiments, the ratio of VEGF PA to acidic and/or basic PAs in a nanofiber determines the mechanical characteristics (e.g., liquid or gel) of the nanofiber material and under what conditions the material will adopt various characteristics (e.g., gelling upon exposure to physiologic conditions, liquifying upon exposure to physiologic conditions, etc.).

Peptide amphiphile (PA) nanofiber solutions may comprise any suitable combination of PAs. In some embodiments, at least 0.05 mg/mL (e.g., 0.10 mg/ml, 0.15 mg/ml, 0.20 mg/ml, 0.25 mg/ml, 0.30 mg/ml, 0.35 mg/ml, 0.40 mg/ml, 0.45 mg/ml, 0.50 mg/ml, 0.60 mg/ml, 0.70 mg/ml, 0.80 mg/ml, 0.90 mg/ml, 1.0 mg/ml, or more, or ranges therebetween), of the solution is a filler PA (e.g., without a peptide epitope or other nanofiber surface displayed moiety). In some embodiments, at least 0.25 mg/mL of the solution is a filler PA. In some embodiments, a filler PA is a non-bioactive PA molecule having highly charged glutamic acid residues on the terminal end of the molecule (e.g., surface-displayed end). These negatively charged PAs allow for the gelation to take place between nanofibers via ionic crosslinks. In some embodiments, a filler PA is a non-bioactive PA molecule having highly charged lysine acid residues on the terminal end of the molecule (e.g., surface-displayed end). These positively charged PAs allow for the gelation to take place under basic conditions. The filler PAs provide the ability to incorporate other bio-active PAs molecules into the nanofiber matrix while still ensuring the ability of the nanofibers solution to gel. In some embodiments, the solutions are annealed for increased viscosity and stronger gel mechanics. These filler PAs have sequences are described in, for example, U.S. Pat. No. 8,772,228 (e.g., C16-VVVAAAEEE (SEQ ID NO: 7)), which is herein incorporated by reference in its entirety.

In some embodiments, the PA nanofiber described herein exhibit a small cross-sectional diameter (e.g., <25 nm, <20 nm, <15 nm, about 10 nm, etc.). In some embodiments, the small cross-section of the nanofibers (˜10 nm diameter) allows the fibers to permeate the brain parenchyma.

In some embodiments, the PAs and nanofibers described herein (e.g., nano-VEGF) find use in the treatment or prevention of neurodegenerative diseases, polyglutamine diseases, and in particular Spinocerebellar Ataxia Type 1 (SCA1).

In some embodiments, the PAs and nanofibers described herein (e.g., nano-VEGF) find use in the treatment or prevention of neurodegenerative diseases, traumatic or mechanical injury to the central nervous system (CNS), spinal cord or peripheral nervous system (PNS), or other diseases or conditions of the nervous system. Examples of diseases and conditions that may be treated and/or prevented by methods and compositions herein include, but are not limited to, Alzheimer's disease (AD), Parkinson's disease (PD), Huntington's disease (HD), spinocerebellar ataxias, amyotrophic lateral sclerosis (ALS; Lou Gehrig's disease), diffuse Lewy body disease, chorea-acanthocytosis, primary lateral sclerosis, ocular diseases (ocular neuritis), chemotherapy-induced neuropathies (e.g., from vincristine, paclitaxel, bortezomib), diabetes-induced neuropathies and Friedreich's ataxia.

In some embodiments, VEGF PA nanofibers herein find use in the treatment of dementia and dementia-related diseases and conditions. Dementias are diseases that include memory loss and additional intellectual impairment separate from memory. The VEGF PA nanofibers herein are suitable for use in treating patients suffering from memory impairment in all forms of dementia. Dementias are classified according to their cause and include: neurodegenerative dementias (e.g., Alzheimer's, Parkinson's disease, Huntington's disease, Pick's disease), vascular (e.g., infarcts, hemorrhage, cardiac disorders), mixed vascular and Alzheimer's, bacterial meningitis, Creutzfeld-Jacob Disease, multiple sclerosis, traumatic (e.g., subdural hematoma or traumatic brain injury), infectious (e.g., HIV), genetic (down syndrome), toxic (e.g., heavy metals, alcohol, some medications), metabolic (e.g., vitamin B12 or folate deficiency), CNS hypoxia, Cushing's disease, psychiatric (e.g., depression and schizophrenia), and hydrocephalus.

The condition of memory impairment is manifested by impairment of the ability to learn new information and/or the inability to recall previously learned information. The present invention includes methods for dealing with memory loss separate from dementia, including mild cognitive impairment (MCI) and age-related cognitive decline. The present invention includes methods of treatment for memory impairment as a result of disease. Memory impairment is a primary symptom of dementia and can also be a symptom associated with such diseases as Alzheimer's disease, schizophrenia, Parkinson's disease, Huntington's disease, Pick's disease, Creutzfeld-Jakob disease, HIV, cardiovascular disease, and head trauma as well as age-related cognitive decline. The VEGF PA nanofibers herein are suitable for use in the treatment of memory impairment due to, for example, Alzheimer's disease, multiple sclerosis, amylolaterosclerosis (ALS), multiple systems atrophy (MSA), schizophrenia, Parkinson's disease, Huntington's disease, Pick's disease, Creutzfeld-Jakob disease, depression, aging, head trauma, stroke, spinal cord injury, CNS hypoxia, cerebral senility, diabetes associated cognitive impairment, memory deficits from early exposure of anesthetic agents, multiinfarct dementia and other neurological conditions including acute neuronal diseases, as well as HIV and cardiovascular diseases.

The VEGF PA nanofibers herein are also suitable for use in the treatment of a class of disorders known as polyglutamine-repeat diseases. These diseases share a common pathogenic mutation. The expansion of a CAG repeat, which encodes the amino acid glutamine, within the genome leads to production of a mutant protein having an expanded polyglutamine region. For example, Huntington's disease has been linked to a mutation of the protein huntingtin. In individuals who do not have Huntington's disease, huntingtin has a polyglutamine region containing about 8 to 31 glutamine residues. For individuals who have Huntington's disease, huntingtin has a polyglutamine region with over 37 glutamine residues. Aside from Huntington's disease (HD), other known polyglutamine-repeat diseases and the associated proteins include dentatorubral-pallidoluysian atrophy, DRPLA (atrophin-1); spinocerebellar ataxia type-1 (ataxin-1); spinocerebellar ataxia type-2 (ataxin-2); spinocerebellar ataxia type-3 (also called Machado-Joseph disease or MJD) (ataxin-3); spinocerebellar ataxia type-6 (alpha 1a-voltage dependent calcium channel); spinocerebellar ataxia type-7 (ataxin-7); and spinal and bulbar muscular atrophy (SBMA, also know as Kennedy disease).

In some embodiments, compositions and methods herein find use in inducing regrowth of blood vessels for the treatment of vascular diseases like critical limb ischemia. In some embodiments, compositions and methods herein find use in the treatment or prevention of SCA1. compositions and methods herein find use in activating cell signaling in vitro (e.g., as a cell culture manipulation tool).

In some embodiments, the VEGF PA nanofiber compositions herein are formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. In some embodiments, VEGF PA nanofiber compositions are administered parenterally. In some embodiments, parenteral administration is by intrathecal administration, intracerebroventricular administration, or intraparenchymal administration.

The VEGF PA nanofiber compositions herein can be administered as the sole active agent or in combination with other pharmaceutical agents such as other agents used in the treatment of neurological diseases and conditions, e.g., nicotinic .alpha.-7 agonists, PDE4 inhibitors, other PDE10 inhibitors, calcium channel blockers, muscarinic m1 and m2 modulators, adenosine receptor modulators, ampakines, NMDA-R modulators, mGluR modulators, dopamine modulators, serotonin modulators, canabinoid modulators, and cholinesterase inhibitors (e.g., donepezil, rivastigimine, and galanthanamine). Drugs suitable in combination with the compounds of the present invention include, but are not limited to Clozaril, Zyprexa, Risperidone, Seroquel, Lithium, Zyprexa, Depakote, Levodopa, Parlodel, Permax, Mirapex, Tasmar, Contan, Kemadin, Artane, Cogentin, Reminyl, Akatinol, Neotropin, Eldepryl, Estrogen Cliquinol, Thioridazine, Haloperidol, Risperidone, Cognex, Aricept, and Exelon, Dilantin, Luminol, Tegretol, Depakote, Depakene, Zarontin, Neurontin, Barbita, Solfeton, Felbatol, Detrol, Ditropan XL, OxyContin, Betaseron, Avonex, Azothioprine, Methotrexate, Copaxone, Amitriptyline, Imipramine, Despiramine, Nortriptyline, Paroxetine, Fluoxetine, Setraline, Terabenazine, Haloperidol, Chloropromazine, Thioridazine, Sulpride, Quetiapine, Clozapine, Risperidone, etc.

### EXPERIMENTAL

### Materials and Methods

### Human Brain Immunohistochemistry

SCA1 patient (n=5) and age-matched (between the ages of 30-55 years) healthy control cerebella (n=4) were provided as paraffin blocks (Edamakanti et al., 2018; herein incorporated by reference in its entirety). The Pathology Core Facility at Northwestern University performed immunohistochemistry for VEGF (Anti-VEGF ab39250; Abcam 1:100, Cambridge, UK) on 5 μm-thick human cerebellar tissues. Optical density analysis was performed with background subtraction using ImageJ using color deconvolution method (Ruifrok and Johnston, 2001; herein incorporated by reference in its entirety).

### Mouse Lines

SCA1154Q/2Q knock-in mice were generated as described previously (Watase et al., 2002b; herein incorporated by reference in its entirety). These mice express a pathogenic polyglutamine tract of 154 repeats from one allele; the other allele expresses a normal mouse Atxnl with two repeats (normal human alleles range from 6 to 44 polyglutamine repeats) (Quan et al., 1995; Servadio et al., 1995; Goldfarb et al., 1996; herein incorporated by reference in their entireties). Initially generated on a C57BL/6J-129SvEv mixed genetic background, the mice were backcrossed more than ten generations with C57BL/6J mice to eliminate any background effects. Animal experiments were performed in compliance with National Institutes of Health's Guide for the Care and Use of Laboratory Animals and the Northwestern University Institutional Animal Care and Use Committee. All experiments were performed by investigators blinded to the genotype using age-matched controls and littermates. Since the phenotype of SCA1 knock-in mice does not differ significantly between male and female mice (Watase et al., 2002a; Cvetanovic et al., 2011; Fryer et al., 2011; Ruegsegger et al., 2016a; herein incorporated by reference in their entireties), mice were not separated by sex.

### Nano-VEGF Synthesis

Nano-VEGF was engineered by linking to the N terminus of a VEGF peptide (designed to mimic VEGF residues 17-25), a K3G sequence to promote solubility, and a V2A2 β-domain followed by a C16 alkyl chain to promote self-assembly (Webber et al., 2011; herein incorporated by reference in its entirety). The peptides were synthesized using 9-fluorenyl methoxycarbonyl (Fmoc) solid-phase peptide synthesis with rink amide 4-methylbenzhydrylamine resin (EMD Millipore). The synthesis was performed on a Liberty automated microwave peptide synthesizer (CEM Corp, Matthews, N.C.) at Northwestern's Peptide Synthesis Core. Fmoc groups were removed with 30% 4-methylpiperidine and 0.1 M hydroxybenzotriazole (HOBt) in N,N-dimethylformamide (DMF) at 75° C. for 3-4 min for each amino acid or palmitic acid (4 equiv.). The peptides were coupled at 75° C. for 5-10 min using 4 equiv. of O-benzotriazole-N, N, N′, N′-tetramethyluronium hexafluorophosphate (HBTU), and 8 equiv. of N,N-diisopropylethylamine (DIEA). The newly synthesized amphiphilic peptides were cleaved from the resin with a 95:2.5:2.5 trifluoroacetic acid (TFA)/triisopropylsilane (TIPS)/water mixture for 3-4 h. Rotary evaporation and precipitation in cold diethyl ether yielded the crude peptide mixture. Crude peptides were purified by HPLC on a C18 Phenomenex Jupiter column in a water-acetonitrile gradient containing 0.1% v/v TFA. Pure fractions were collected and identified using ESI-MS. The combined fractions were subjected to rotary evaporation to remove volatile solvents; they were then frozen and lyophilized to dryness. The purity of the final lyophilized solid was verified by liquid chromatography mass spectrometry (LC-MS).

Delivery of Nano-VEGF and Recombinant VEGF with Experimental Time Course

To test for behavioral and pathological improvement mice were treated for a two-week period starting at 24 weeks. Nano-VEGF, mouse rVEGF (R&D systems, Minneapolis, Minn.) or artificial CSF (sham controls) were delivered using the intracerebroventricular route (into the right lateral ventricle with stereotaxic coordinates: A/P −0.5 mm, M/L −1.1 mm, D/V −2.5 mm). using osmotic ALZET pumps; model #1002, Durect) (Cvetanovic et al., 2011; herein incorporated by reference in its entirety). Behavioral assays were performed at 26 weeks; while biochemical and histochemical analysis was performed at 27 weeks (after euthanasia). For electrophysiological experiments, we used 8-10 week-old mice for pump placement (with a similar two-week treatment regimen) since it is difficult to generate good electrophysiological traces from older mice.

### Behavioral Analyses

Rotating Rod Assay: 26-week-old mice (post-treatment) were placed on a rotating rod apparatus (Ugo Basile) that accelerates linearly from 4 to 40 rotations per minute over a 5-minute period. Mice were subjected to four trials per day for four consecutive days, each trial lasting to a maximum of ten minutes, with at least ten minutes of rest between each trial. The average performances for each day were plotted, and statistical differences between the different groups were analyzed using repeated measures two-way ANOVA (followed by Tukey's honest significant difference post-hoc test for multiple comparisons). All statistical analyses here and elsewhere were performed using GraphPad Prism 6 software (GraphPad Software, La Jolla Calif. USA). Data were considered significant for p<0.05. Data are expressed as mean±SEM.

Gait Analysis: Mice were placed on a transparent treadmill belt connected to a high-speed digital video camera (Digigait© system; Mouse Specifics, Inc.). The speed of the treadmill was set to the minimum of 5 cm/s up to maximum tolerated speed (maxed out at 24 cm/s). The performance of mice on the treadmill at different speeds was recorded with digital video camera.

### Mouse Immunohistochemistry

27-week-old mice (post treatment and post behavioral analysis) were anesthetized by isoflurane inhalation and were perfused with ice-cold PBS, followed by 4% paraformaldehyde in PBS (Israeli et al., 2016; herein incorporated by reference in its entirety). The brains were post-fixed in 4% paraformaldehyde for 24 hrs, followed by tissue saturation in 30% sucrose for 48-72 hrs. Frozen brain blocks were prepared in OCT (optimum cutting temperature) freezing medium and sagittally sectioned at 50 μm thickness using a cryostat (Leica). Immunohistochemistry was performed (Venkatraman et al., 2014; herein incorporated by reference in its entirety). Brain tissues were blocked and permeabilized with 5% Normal donkey serum in 1×TBS with 0.25% Triton-X 100 (TBST) for 2 hours at room temperature. Slices were then incubated with primary antibodies for 48-72 hours in 4° C. After three washes in TBST, signals were detected using according secondary antibodies for 2 hours at RT. Brain slices were counterstained with DAPI (Invitrogen) and mounted with PVA (Polyvinyl alcohol)-DABCO mounting medium.

At least three mice were studied per experimental group and performed analysis on 3-5 sections per mouse. Images were taken as Z-stack images using the Leica TCS SP5 confocal microscope (Cvetanovic et al., 2011). To observe the morphology of Purkinje cells, calbindin staining (mouse anti-Calbindin-D-28K (C9848, Sigma, 1:2000) was performed. To quantify dendritic length, the calbindin-stained PCs were traced and the length of the PC dendrites was measured from the proximal end of the soma to the distal end of the dendrites (using ImageJ 1.46r Software; National Institutes of Health, Bethesda, Md., USA). The vascular network was visualized by staining for Collagen IV (rabbit anti-Collagen IV (ab19808, Abcam, 1:400). Average vessel length and branching index was plotted and analyzed by assessing the variation in foreground and background pixel mass densities across images (using the open source AngioTool software (angiotool.nci.nih.gov; Zudaire et al., 2011; herein incorporated by reference in its entirety). Statistical differences for dendritic length and microvasculature parameters were compared using one-way ANOVA followed by Tukey's post-hoc test.

To quantify the percentage of cells with ataxin-1 inclusions in the CA1 regions of the hippocampus, brain sections were stained for ataxin-1 (mouse anti-Ataxin-1 11NQ clone N76/3; NeuroMab, 1:1000) and counterstained with DAPI. The number of cells with ataxin-1 inclusions in each defined region of interest was normalized to the number of cells stained with DAPI only. Statistical analysis was performed using unpaired t test. In all cases, the secondary antibodies were generated in donkey (donkey anti-mouse Alexa Fluor®647 and donkey anti-rabbit Alexa Fluor® 488; both at a dilution of 1:500 Invitrogen,).

### Western Blot Analysis

Proteins were extracted from cerebellar tissues using radioimmunoprecipitation assay (RIPA) buffer (50 mM Tris-HCl, pH 8.0, 150 mM NaCl, 1 mM EDTA, 10 mM NaF, 0.5% Sodium deoxycholate, 1% Triton-X 100, 0.1% SDS, protease inhibitors (Sigma)), and resolved on 7.5% SDS-PAGE gel. Densitometric analysis was evaluated using ImageJ 1.46r software (National Institutes of Health, Bethesda, Md., USA). The following primary antibodies were used: mouse anti-Ataxin-1 (11NQ clone N76/3; NeuroMab, 1:1000); rabbit anti-Claudin 5 (34-1600, Thermo Fisher Scientific 1:200); rabbit anti-Occludin (71-1500, Thermo Fisher Scientific, 1:200); mouse anti-ZO-1 (33-9100, Thermo Fisher Scientific, 1:100), and mouse anti-beta Actin (A2228, Sigma, 1:5000). The secondary antibodies used were anti-rabbit IgG (H+L) HRP (W4011, Promega, 1:5000) or anti-mouse IgG (H+L) HRP (W4021, Promega, 1:5000) secondary antibodies (visualized with ECL substrate (GE); film densitometric analysis was evaluated using ImageJ 1.46r software (National Institutes of Health, Bethesda, Md., USA). Statistical differences were compared using one-way ANOVA followed by Tukey's post-hoc test.

### Phospho-VEGFR2 ELISA

Phospho-VEGFR2 ELISA was performed using PathScan® Phospho-VEGFR2 (Ty1175) Sandwich ELISA kit (7335, Cell Signaling), according to manufacturer's instruction. Cerebellar lysates were prepared in 2× cell lysis buffer, and undiluted tissue lysates were incubated in 96-well plates coated with phosphor-VEGFR2 primary antibody overnight at 4° C. Wells were washed several times, and detection antibody was added to the well and incubated for 1 hr at 37C and with HRP-conjugated secondary antibody for 30 min at 37° C. Signals were detected with 3,3′,5,5′-Tetramethylbenzidine (TMB) substrate and sulfuric acid were added to stop the reaction. Absorbance was read at 450 nm within 30 min after stopping the reaction.

### Electrophysiological Slice Recordings

Mice were anesthetized with isoflurane and killed by decapitation. The brains were removed from the skull and placed in warm (30-33° C.) artificial cerebrospinal fluid (ACSF) containing (in mM): 125 NaCl, 25 NaHCO₃, 2.5 KCl, 1.25 NaH2PO4, 0.5 CaCl₂), 7 MgCl2, 75 sucrose and 25 glucose, equilibrated with 95% O2 and 5% CO2 (pH 7.4). 300 μm thick para-sagittal cerebellar slices were cut using a vibratome (Leica VT1200) and stored in the same solution at 34° C. for 15 min and then at 20-22° C. until used (<6 hours). For electrophysiological recordings, the slices were transferred to a chamber bathed in physiological ACSF (in mM: 125 NaCl, 25 NaHCO₃, 2.5 KCl, 1.25 NaH2PO3, 25 glucose, 2.0 CaCl₂) and 1 MgCl2, (equilibrated with 95% 02 and 5% CO2) at 30-32° C. Slices were visualized using an upright microscope (Scientifica) equipped with a 40× water-immersion objective (Olympus), oblique illumination, and video microscopy using a digital camera (Q-imaging). Pipettes for cell-attached recordings were pulled from borosilicate glass (Sutter) using a horizontal puller (P97, Sutter). Tip resistances in working solution ranged from 3 to 6 MΩ when filled with physiological ACSF. Electrophysiological recordings were obtained using an Axopatch 200B amplifier, filtered at 20 KHz and acquired at 10 KHz. Data were obtained and visualized using pClamp9 software. Cells were recorded in the I-Clamp fast mode. For all recordings, fast synaptic transmission was blocked by 3 mM kynurenic acid (Sigma) and 0.1 mM picrotoxin (Abcam). Statistical analysis was performed using unpaired t test.

### Results

Nano-VEGF Improves Motor Coordination in SCA1 Mice with Advanced Disease

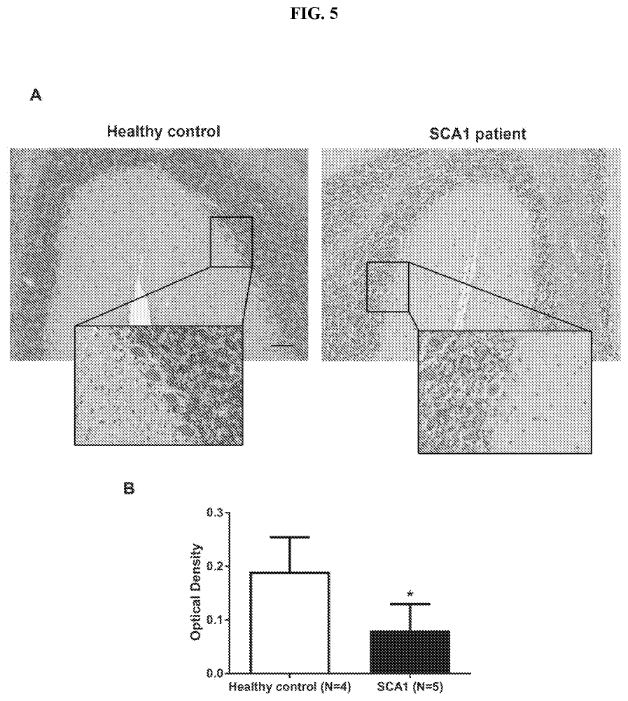

Cerebella of SCA1 knock-in mice show lower VEGF expression than wild-type (Cvetanovic et al., 2011; herein incorporated by reference in its entirety). To establish whether these observations extend to human patients, immunohistochemical analysis was performed on post-mortem cerebella obtained from SCA1 patients and age-matched controls. VEGF staining is clearly reduced in the cerebellar lobules of SCA1 patients (FIGS. 5A and B).

To test the therapeutic benefits of nano-VEGF, SCA1 knock-in mice (SCA1154Q/2Q), which reproduce the human disease remarkably well, were used. Born without symptoms, they develop an adult-onset motor incoordination (around 8 weeks of age) that is steadily progressive and readily quantifiable by the accelerating rotating rod test (Watase et al., 2002b; Cvetanovic et al., 2011; Ruegsegger et al., 2016b; herein incorporated by reference in their entireties). They also develop progressive Purkinje cell (PC) degeneration, and show diminished cerebellar vasculature and PC dendritic arborization. It has been demonstrated previously that recombinant VEGF, delivered over a two-week period starting at 11 weeks of age, improves motor coordination and mitigates these pathological features (Cvetanovic et al., 2011; herein incorporated by reference in its entirety).

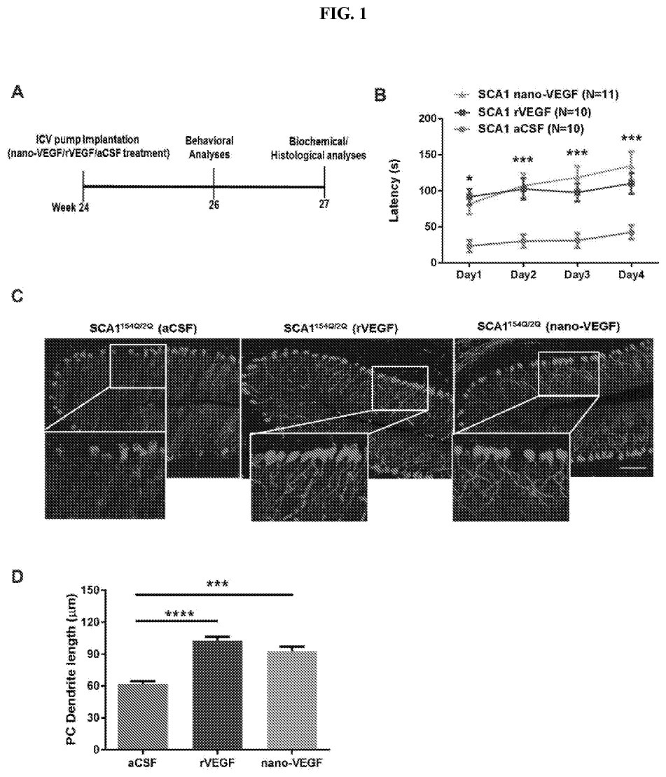

Nano-VEGF was tested against recombinant VEGF (rVEGF) on SCA1 mice at 24 weeks of age, which corresponds to advanced disease in humans. Any improvement in these mice would be a more stringent test of efficacy. The compounds were delivered over a two-week period, rVEGF at a dose of 2.5 μg (dissolved in 100 μl of artificial CSF (aCSF) (Cvetanovic et al., 2011), and nano-VEGF at a dose of 20.0 μg [the bioequivalence of nano-VEGF was determined to be approximately eight times that of rVEGF based on functional studies in a mouse hind-limb ischemia model (Webber et al., 2011; herein incorporated by reference in its entirety). The effects of both forms of VEGF were then evaluated at the end of the delivery period by behavioral testing in the 26th week and neuropathological assessment in the 27th week (FIG. 1A).

Rotarod analysis at 26 weeks showed that SCA1 mice treated with either rVEGF or nano-VEGF performed markedly better than control SCA1 mice treated with artificial CSF (p<0.005) (FIG. 1B; nano-VEGF tended to produce a greater improvement than rVEGF). Gait analysis was performed using a videotaped assessment of gait on a transparent treadmill. SCA1 mice were barely able to walk on the moving treadmill even at low speeds (5 cm/s), but mice treated with nano-VEGF (or rVEGF) could walk and even run when the speed of the treadmill was increased to 17 cm/sec (tested up to 24 cm/s).

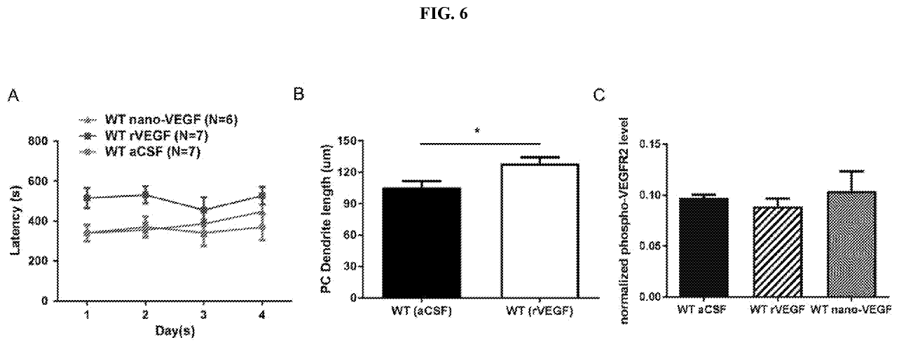

Cerebellar sections from SCA1 mice were stained with calbindin, which specifically stains Purkinje cells and is a standard technique to assess PC neuropathology in SCA1. PCs in treated SCA1 mice had longer dendrites than aCSF-treated controls (FIGS. 1C and 1D). VEGF treatment did not affect the motor performance of wild-type mice, but did lengthen PC dendrites (FIGS. 6A and B).

Nano-VEGF Improves Neuropathological Measures in SCA1 Mice with Advanced Disease

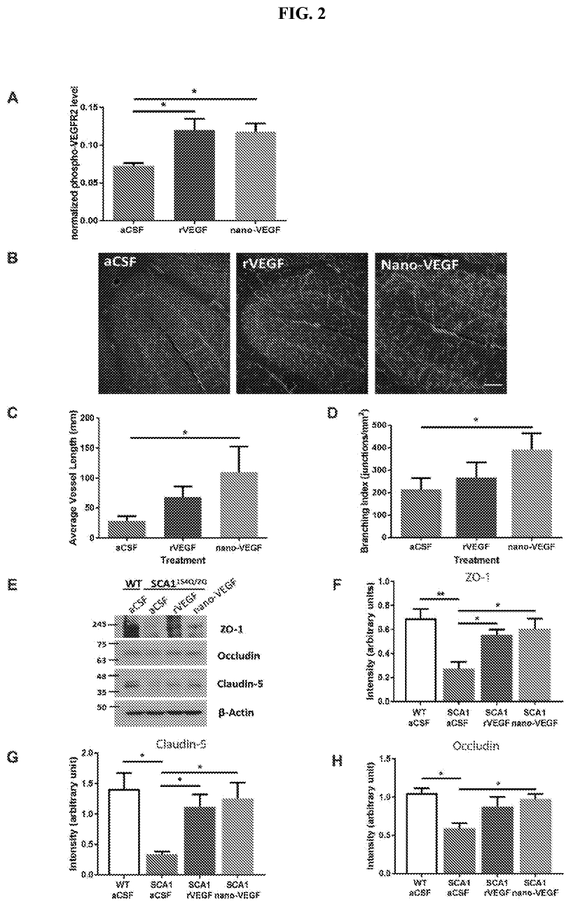

In the nervous system, the effects of VEGF are mediated primarily by the membrane-spanning VEGF receptor 2 (VEGFR2), a tyrosine kinase receptor formed of two monomers. When VEGF binds, the monomers dimerize to cause autophosphorylation of the intracellular tyrosine residues, which activates downstream signaling pathways that mediate its effects (Cross and Claesson-Welsh, 2001; herein incorporated by reference in its entirety). ELISA showed a decrease in the level of VEGFR2 phosphorylation consistent with low levels of VEGF in SCA1 mice compared to controls. This reduction of VEGFR2 phosphorylation is rescued by both rVEGF and nano-VEGF treatment, indicating that nano-VEGF functionally engages VEGF receptors (FIG. 2A). Consistent with the lack of effect on motor performance in wild-type mice, VEGF treatment did not upregulate phosphorylation of VEGFR2 in WT mice, indicating that there is a ceiling effect, and the brain has only so much capacity for VEGF signaling (FIG. 6C).

Low levels of VEGF in SCA1 mice are associated with a pathological reduction in the microvasculature in SCA1 cerebella by day 30, the earliest time tested. This vessel reduction is quantifiable as a decrease in the length and branching of capillaries (Cvetanovic et al., 2011; herein incorporated by reference in its entirety). Mice treated with nano-VEGF showed a greater improvement in vessel length and branching index than mice treated with rVEGF (FIG. 2B-2D). Western blot studies showed that reduced levels of capillary proteins (the tight junction markers ZO-1, Claudin-4, and Occludin (Chiba et al., 2008; Zhong et al., 2008; herein incorporated by reference in their entireties)) in SCA1 mice are restored with VEGF replenishment, and again nano-VEGF was more effective than rVEGF (FIG. 2E-2H).

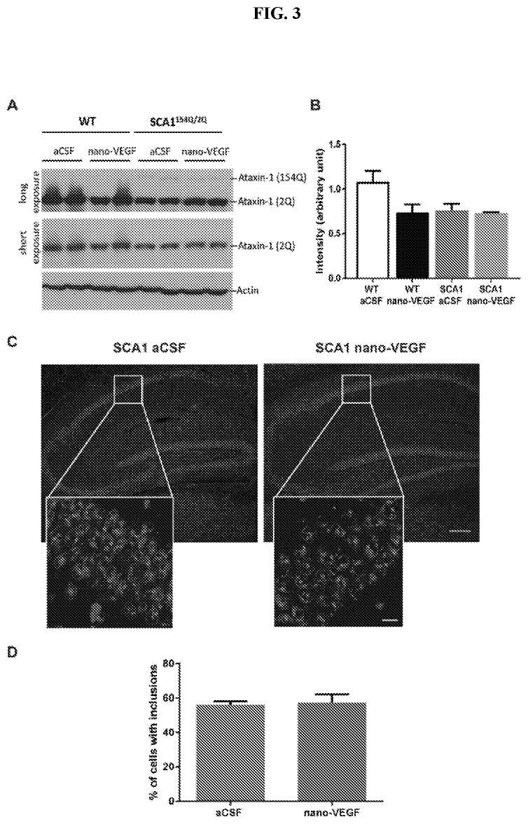

Since the toxicity in SCA1 is caused by accumulation of Ataxin-1, experiments were conducted during development of embodiments herein to determine whether VEGF might exert some effect, perhaps indirect, on protein levels. No difference in Ataxin-1 levels between SCA1 and wild-type mouse brains (FIG. 3A; the western blot is shown at two different exposures: the short exposure reveals ataxin-1 2Q, while the long exposure also reveals ataxin-1 154Q, which is poorly soluble and remains in aggregates). Finally, one of the hallmarks of SCA1 pathology is an aggregation of Ataxin-1 into nuclear inclusions in relatively spared brain regions such as the hippocampus. 27-week-old mice treated with VEGF show no reduction in the levels of inclusions (FIGS. 3B and C). These results indicate that VEGF does not exert its beneficial effects by improving Ataxin-1 proteostasis.

Nano-VEGF Treatment Improves the Firing of Purkinje Neurons in Mice with Advanced SCA1

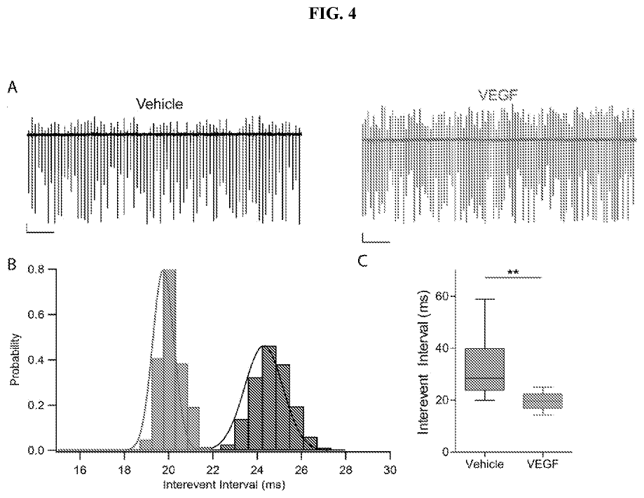

To study the physiological effects of VEGF treatment on Purkinje cells, which is very difficult to do in older mice because of the poorer quality of slice cultures, a cohort of younger SCA1 mice from 8-10 weeks were also treated with nano-VEGF before performing electrophysiology. Purkinje cells provide the only output of the cerebellar cortex and transmit the integrated activity of the cortex to the cerebellar nuclei. In SCA1 mice, PCs show less spontaneous firing not only because of their own pathology but also because of a decrease in climbing fiber (CF) excitatory postsynaptic currents (EPSCs) (Dell'Orco et al., 2015; Ruegsegger et al., 2016b; herein incorporated by reference in their entireties) and an increase in GABA-ergic inhibition from basket cells (Edamakanti et al., 2018; herein incorporated by reference in its entirety). Nano-VEGF-treated mice showed significantly faster and more regular firing compared with non-treated SCA1 mice, in keeping with the behavioral improvement (FIG. 4A-C).

Nano-VEGF thus activates VEGF signaling receptors, improves pathology and enhances the motor ability of SCA1 mice. The disease-modifying effects are quite remarkable, given that the treatment regimen was initiated only after neurodegeneration was well-established.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

Any patents and publications referenced herein are herein incorporated by reference in their entireties.

### REFERENCES

The following references, some of which are cited above, are herein incorporated by reference in their entireties.
Bottros M M, Christo P J. Current perspectives on intrathecal drug delivery. Journal of Pain Research 2014; 7: 615-26.
Caballero B, Sherman S J, Falk T. Insights into the Mechanisms Involved in Protective Effects of VEGF-B in Dopaminergic Neurons. Parkinson's disease 2017; 2017: 4263795.
Carmeliet P. VEGF as a key mediator of angiogenesis in cancer. Oncology 2005; 69 Suppl 3: 4-10.
Chiba H, Osanai M, Murata M, Kojima T, Sawada N. Transmembrane proteins of tight junctions. Biochimica et Biophysica Acta (BBA)—Biomembranes 2008; 1778(3): 588-600.
Cross M J, Claesson-Welsh L. FGF and VEGF function in angiogenesis: signalling pathways, biological responses and therapeutic inhibition. Trends in pharmacological sciences 2001; 22(4): 201-7.
Cvetanovic M, Patel J M, Marti H H, Kini A R, Opal P. Vascular endothelial growth factor ameliorates the ataxic phenotype in a mouse model of spinocerebellar ataxia type 1. Nature medicine 2011; 17(11): 1445-7.
D'Andrea L D, Iaccarino G, Fattorusso R, Sorriento D, Carannante C, Capasso D, et al. Targeting angiogenesis: structural characterization and biological properties of a de novo engineered VEGF mimicking peptide. Proceedings of the National Academy of Sciences of the United States of America 2005; 102(40): 14215-20.
De Rosa L, Finetti F, Diana D, Di Stasi R, Auriemma S, Romanelli A, et al. Miniaturizing VEGF: Peptides mimicking the discontinuous VEGF receptor-binding site modulate the angiogenic response. Sci Rep 2016; 6: 31295.
Dell'Orco J M, Wasserman A H, Chopra R, Ingram M A, Hu Y S, Singh V, et al. Neuronal Atrophy Early in Degenerative Ataxia Is a Compensatory Mechanism to Regulate Membrane Excitability. J Neurosci 2015; 35(32): 11292-307.
Diana D, Ziaco B, Colombo G, Scarabelli G, Romanelli A, Pedone C, et al. Structural determinants of the unusual helix stability of a de novo engineered vascular endothelial growth factor (VEGF) mimicking peptide. Chemistry (Weinheim an der Bergstrasse, Germany) 2008; 14(14): 4164-6.
Echeverria V, Barreto G E, Avila-Rodrigueze M, Tarasov V V, Aliev G. Is VEGF a Key Target of Cotinine and Other Potential Therapies Against Alzheimer Disease? Current Alzheimer research 2017; 14(11): 1155-63.
Edamakanti C R, Do J, Didonna A, Martina M, Opal P. Mutant ataxin1 disrupts cerebellar development in spinocerebellar ataxia type 1. The Journal of clinical investigation 2018; 128(6): 2252-65.
Fryer J D, Yu P, Kang H, Mandel-Brehm C, Carter A N, Crespo-Barreto J, et al. Exercise and genetic rescue of SCA1 via the transcriptional repressor Capicua. Science 2011; 334(6056): 690-3.
Goldfarb L G, Vasconcelos O, Platonov F A, Lunkes A, Kipnis V, Kononova S, et al. Unstable triplet repeat and phenotypic variability of spinocerebellar ataxia type 1. Annals of neurology 1996; 39(4): 500-6.
Holmes D I R, Zachary I. The vascular endothelial growth factor (VEGF) family: angiogenic factors in health and disease. Genome Biology 2005; 6(2): 209-.
Iadecola C. The Neurovascular Unit Coming of Age: A Journey through Neurovascular Coupling in Health and Disease. Neuron 2017; 96(1): 17-42.
Israeli E, Dryanovski D I, Schumacker P T, Chandel N S, Singer J D, Julien J P, et al. Intermediate filament aggregates cause mitochondrial dysmotility and increase energy demands in giant axonal neuropathy. Hum Mol Genet 2016; 25(11): 2143-57.
Jin K, Zhu Y, Sun Y, Mao X O, Xie L, Greenberg D A. Vascular endothelial growth factor (VEGF) stimulates neurogenesis in vitro and in vivo. Proceedings of the National Academy of Sciences of the United States of America 2002; 99(18): 11946-50.
Quan F, Janas J, Popovich B W. A novel CAG repeat configuration in the SCA1 gene: implications for the molecular diagnostics of spinocerebellar ataxia type 1. Hum Mol Genet 1995; 4(12): 2411-3.
Ruegsegger C, Stucki D M, Steiner S, Angliker N, Radecke J, Keller E, et al. Impaired mTORC1-Dependent Expression of Homer-3 Influences SCA1 Pathophysiology. Neuron 2016a; 89(1): 129-46.
Ruegsegger C, Stucki David M, Steiner S, Angliker N, Radecke J, Keller E, et al. Impaired mTORC1-Dependent Expression of Homer-3 Influences SCA1 Pathophysiology. Neuron 2016b; 89(1): 129-46.
Ruifrok A C, Johnston D A. Quantification of histochemical staining by color deconvolution. Analytical and quantitative cytology and histology 2001; 23(4): 291-9.
Servadio A, Koshy B, Armstrong D, Antalffy B, Orr H T, Zoghbi H Y. Expression analysis of the ataxin-1 protein in tissues from normal and spinocerebellar ataxia type 1 individuals. Nat Genet 1995; 10(1): 94-8.
Silva G A, Czeisler C, Niece K L, Beniash E, Harrington D A, Kessler J A, et al. Selective differentiation of neural progenitor cells by high-epitope density nanofibers. Science 2004; 303(5662): 1352-5.
Storkebaum E, Lambrechts D, Dewerchin M, Moreno-Murciano M-P, Appelmans S, Oh H, et al. Treatment of motoneuron degeneration by intracerebroventricular delivery of VEGF in a rat model of ALS. Nat Neurosci 2005; 8(1): 85-92.
Thorne R G, Frey W H, 2nd. Delivery of neurotrophic factors to the central nervous system: pharmacokinetic considerations. Clinical pharmacokinetics 2001; 40(12): 907-46.
Venkatraman A, Hu Y S, Didonna A, Cvetanovic M, Krbanjevic A, Bilesimo P, et al. The histone deacetylase HDAC3 is essential for Purkinje cell function, potentially complicating the use of HDAC inhibitors in SCA1. Hum Mol Genet 2014; 23(14): 3733-45.
Verheul H M, van Erp K, Homs M Y, Yoon G S, van der Groep P, Rogers C, et al. The relationship of vascular endothelial growth factor and coagulation factor (fibrin and fibrinogen) expression in clear cell renal cell carcinoma. Urology 2010; 75(3): 608-14.
Wang Y, Duan W, Wang W, Di W, Liu Y, Liu Y, et al. scAAV9-VEGF prolongs the survival of transgenic ALS mice by promoting activation of M2 microglia and the PI3K/Akt pathway. Brain research 2016; 1648(Pt A): 1-10.
Watase K, Weeber E J, Xu B, Antalffy B, Yuva-Paylor L, Hashimoto K, et al. A long CAG repeat in the mouse Sca1 locus replicates SCA1 features and reveals the impact of protein solubility on selective neurodegeneration. Neuron 2002a; 34(6): 905-19.
Watase K, Weeber E J, Xu B, Antalffy B, Yuva-Paylor L, Hashimoto K, et al. A Long CAG Repeat in the Mouse Sca1 Locus Replicates SCA1 Features and Reveals the Impact of Protein Solubility on Selective Neurodegeneration. Neuron 2002b; 34(6): 905-19.
Webber M J, Tongers J, Newcomb C J, Marquardt K T, Bauersachs J, Losordo D W, et al. Supramolecular nanostructures that mimic VEGF as a strategy for ischemic tissue repair. Proceedings of the National Academy of Sciences of the United States of America 2011; 108(33): 13438-43.
Wiesmann C, Fuh G, Christinger H W, Eigenbrot C, Wells J A, de Vos A M. Crystal structure at 1.7 A resolution of VEGF in complex with domain 2 of the Flt-1 receptor. Cell 1997; 91(5): 695-704.
Yang R, Ogasawara A K, Zioncheck T F, Ren Z, He G W, DeGuzman G G, et al. Exaggerated hypotensive effect of vascular endothelial growth factor in spontaneously hypertensive rats. Hypertension (Dallas, Ill.: 1979) 2002; 39(3): 815-20.
Zhong Z, Deane R, Ali Z, Parisi M, Shapovalov Y, O'Banion M K, et al. ALS-causing SOD1 mutants generate vascular changes prior to motor neuron degeneration. Nat Neurosci 2008; 11(4): 420-2.
Zou J, Chen Z, Wei X, Chen Z, Fu Y, Yang X, et al. Cystatin C as a potential therapeutic mediator against Parkinson's disease via VEGF-induced angiogenesis and enhanced neuronal autophagy in neurovascular units. Cell death & disease 2017; 8(6): e2854.

1. A peptide amphiphile nanofiber comprising:
(a) a bioactive peptide amphiphile comprising:
(i) a hydrophobic non-peptidic segment;
(ii) a β-sheet-forming peptide segment;
(iii) a charged peptide segment; and
(iv) a VEGF peptide comprising at least 50% sequence identity with KLTWQELYQLKYKGI (SEQ ID NO:1); and
(b) a charged peptide amphiphile comprising:
(i) a hydrophobic non-peptidic segment;
(ii) a β-sheet-forming peptide segment; and
(iii) a charged peptide segment.

2. The peptide amphiphile nanofiber of claim 1, wherein the charged peptide amphiphile does not comprise a bioactive peptide.

3. The peptide amphiphile nanofiber of claim 1, wherein the hydrophobic non-peptidic segment of the bioactive peptide amphiphile and the charged peptide amphiphile comprises an acyl chain.

4. The peptide amphiphile nanofiber of claim 3, wherein the acyl chain comprises C6-C20.

5. The peptide amphiphile nanofiber of claim 4, wherein the acyl chain comprises C16.

6. The peptide amphiphile nanofiber of claim 1, wherein the β-sheet-forming peptide segment of the bioactive peptide amphiphile and the charged peptide amphiphile comprises AAAVVV (SEQ ID NO: 2) or AAVV (SEQ ID NO: 3).

10. The peptide amphiphile nanofiber of claim 1, wherein the charged peptide segment of the bioactive peptide amphiphile is an acidic peptide segment.

11. The peptide amphiphile nanofiber of claim 10, wherein the acidic peptide segment of the bioactive peptide amphiphile comprises Glu (E) and/or Asp (D) residues.

12. The peptide amphiphile nanofiber of claim 11, wherein the acidic peptide segment of the bioactive peptide amphiphile comprises is 2-7 amino acids in length with 50% or more amino acids selected from Glu (E) and/or Asp (D) residues.

13. The peptide amphiphile nanofiber of claim 12, wherein the acidic peptide segment of the bioactive peptide amphiphile comprises EEE.

14. The peptide amphiphile nanofiber of claim 1, wherein the charged peptide segment of the bioactive peptide amphiphile is a basic peptide segment.

15. The peptide amphiphile nanofiber of claim 14, wherein the basic peptide segment of the bioactive peptide amphiphile comprises one or more lysine (K), histidine (H), and/or arginine (R) residues.

16. The peptide amphiphile nanofiber of claim 15, wherein the basic peptide segment of the bioactive peptide amphiphile comprises is 2-7 amino acids in length with 50% or more Lys (K) residues.

17. The peptide amphiphile nanofiber of claim 16, wherein the basic peptide segment of the bioactive peptide amphiphile comprises KKK.

18. The peptide amphiphile nanofiber of claim 1, wherein the charged peptide segment of the charged peptide amphiphile is an acidic peptide segment.

19. The peptide amphiphile nanofiber of claim 18, wherein the acidic peptide segment of the charged peptide amphiphile comprises Glu (E) and/or Asp (D) residues.

20. The peptide amphiphile nanofiber of claim 19, wherein the acidic peptide segment of the charged peptide amphiphile comprises is 2-7 amino acids in length with 50% or more amino acids selected from Glu (E) and/or Asp (D) residues.

21. The peptide amphiphile nanofiber of claim 20, wherein the acidic peptide segment of the charged peptide amphiphile comprises EEE.

22. The peptide amphiphile nanofiber of claim 1, wherein the charged peptide segment of the charged peptide amphiphile is a basic peptide segment.

23. The peptide amphiphile nanofiber of claim 22, wherein the basic peptide segment of the charged peptide amphiphile comprises one or more lysine (K), histidine (H), and/or arginine (R) residues.

24. The peptide amphiphile nanofiber of claim 23, wherein the basic peptide segment of the charged peptide amphiphile comprises is 2-7 amino acids in length with 50% or more Lys (K) residues.

25. The peptide amphiphile nanofiber of claim 24, wherein the basic peptide segment of the charged peptide amphiphile comprises KKK.

26. The peptide amphiphile nanofiber of claim 1, wherein the VEGF peptide comprises at least 70% sequence identity with SEQ ID NO: 1.

27. The peptide amphiphile nanofiber of claim 18, wherein the VEGF peptide comprises SEQ ID NO: 1.

28. The peptide amphiphile nanofiber of claim 1, wherein the peptide amphiphile nanofiber comprises 5%-75% by mass bioactive peptide amphiphile and 25% to 95% (by mass basic peptide amphiphile, and wherein the nanofiber forms a gel under basic conditions.

29. The peptide amphiphile nanofiber of claim 1, wherein the peptide amphiphile nanofiber comprises 75-99% by mass bioactive peptide amphiphile and 1% to 25% by mass basic peptide amphiphile, and wherein the nanofiber is a liquid under basic conditions.

30. The peptide amphiphile nanofiber of claim 1, wherein the peptide amphiphile nanofiber comprises 1-20% by mass bioactive peptide amphiphile and 80-99% by mass acidic peptide amphiphile, and wherein the nanofiber forms a gel under acidic conditions.

31. The peptide amphiphile nanofiber of claim 1, wherein the peptide amphiphile nanofiber comprises 20-80% by mass bioactive peptide amphiphile and 20-80% by mass acidic peptide amphiphile, and wherein the nanofiber forms a gel under neutral conditions.

32. The peptide amphiphile nanofiber of claim 1, wherein the peptide amphiphile nanofiber comprises 80-99% by mass bioactive peptide amphiphile and 1-20% by mass acidic peptide amphiphile, and wherein the nanofiber is a liquid under acidic conditions.

33. A peptide amphiphile nanofiber comprising:
(a) a bioactive peptide amphiphile comprising:
(i) a hydrophobic non-peptidic segment comprising a C6-C20 acyl chain;
(ii) a β-sheet-forming peptide segment comprising AAAVVV (SEQ ID NO: 2) or AAVV (SEQ ID NO: 3);
(iii) a charged peptide segment, wherein the charged peptide segment comprises:
(A) an acidic peptide segment comprising EEE, EED, EDE, DEE, EDD, DED, DDE, or DDD; or
(B) a basic peptide segment comprising 2-7 or more lysine (K), histidine (H), and/or arginine (R) residues; and
(iv) a VEGF peptide comprising at least 50% sequence identity with KLTWQELYQLKYKGI (SEQ ID NO:1); and
(b) a charged peptide amphiphile comprising:
(i) a hydrophobic non-peptidic segment comprising a C₆-C₂₀ acyl chain;
(ii) a β-sheet-forming peptide segment comprising AAAVVV (SEQ ID NO: 2) or AAVV (SEQ ID NO: 3); and
(iii) a charged peptide segment, wherein the charged peptide segment comprises:
(A) an acidic peptide segment comprising EEE, EED, EDE, DEE, EDD, DED, DDE, or DDD; or
(B) a basic peptide segment comprising 2-7 or more lysine (K), histidine (H), and/or arginine (R) residues.

34. The peptide amphiphile nanofiber of one of claims 1-33 comprising a linker segment between the charged peptide segment and the bioactive peptide segment.

35. The peptide amphiphile nanofiber of claim 33, wherein the linker segment comprises 1-3 glycine (G) residues.

36. The peptide amphiphile nanofiber of one of claims 1-35 comprising a bioactive peptide amphiphile with at least 70% sequence identity with one selected from one of C16-V3A3E3G-KLTWQELYQLKYKGI (SEQ ID NO: 8), C16-V3A3E4G-KLTWQELYQLKYKGI (SEQ ID NO: 9), C16-V2A2E2G-KLTWQELYQLKYKGI (SEQ ID NO: 10), C16-V2A2E4G-KLTWQELYQLKYKGI (SEQ ID NO: 11), C16-V2A2E4G4-KLTWQELYQLKYKGI (SEQ ID NO: 12), C16-A2G2E4G-KLTWQELYQLKYKGI (SEQ ID NO: 13), C16-VEVA2E4G-KLTWQELYQLKYKGI (SEQ ID NO: 14), C16-V2A2K3G-KLTWQELYQLKYKGI (SEQ ID NO: 15), C16-V2A2K3G4-KLTWQELYQLKYKGI (SEQ ID NO: 16), and C16-V3A3K3G-KLTWQELYQLKYKGI (SEQ ID NO: 17).

37. The peptide amphiphile nanofiber of claim 36 comprising a bioactive peptide amphiphile selected from one of C16-V3A3E3G-KLTWQELYQLKYKGI (SEQ ID NO: 8), C16-V3A3E4G-KLTWQELYQLKYKGI (SEQ ID NO: 9), C16-V2A2E2G-KLTWQELYQLKYKGI (SEQ ID NO: 10), C16-V2A2E4G-KLTWQELYQLKYKGI (SEQ ID NO: 11), C16-V2A2E4G4-KLTWQELYQLKYKGI (SEQ ID NO: 12), C16-A2G2E4G-KLTWQELYQLKYKGI (SEQ ID NO: 13), C16-VEVA2E4G-KLTWQELYQLKYKGI (SEQ ID NO: 14), C16-V2A2K3G-KLTWQELYQLKYKGI (SEQ ID NO: 15), C16-V2A2K3G4-KLTWQELYQLKYKGI (SEQ ID NO: 16), and C16-V3A3K3G-KLTWQELYQLKYKGI (SEQ ID NO: 17).

38. A method of treating a neurologic condition comprising administering a pharmaceutical composition comprising a peptide amphiphile nanofiber of one of claims 1-37 to a subject suffering from the neurologic condition.

39. The method of claim 38, wherein the neurologic condition is a polyglutamine disease.

40. The method of claim 39, wherein the polyglutamine disease Spinocerebellar Ataxia Type 1.

41. The method of claim 38, wherein the pharmaceutical composition is administered parenterally.

42. The method of claim 41, wherein the pharmaceutical composition is administered by intrathecal administration, intracerebroventricular administration, or intraparenchymal administration.
